(19) United States
(12) Patent Application Publication
(10) Pub. No.: US 2024/0026916 A1
(43) Pub. Date: Jan. 25, 2024

(54) UNIVERSAL FASTENING SYSTEM

(71) Applicant: KIANOR SHAHMOHAMMADI, Palm Desert, CA (US)

(72) Inventor: KIANOR SHAHMOHAMMADI, Palm Desert, CA (US)

(73) Assignee: KIANOR SHAHMOHAMMADI, Palm Desert, CA (US)

(21) Appl. No.: 18/254,173

(22) PCT Filed: Jan. 20, 2022
(86) PCT No.: PCT/IB2022/050477

§ 371 Date: May 24, 2023

(51) Int. Cl. F16B 23/00; B23B 51/00

(52) U.S. Cl. CPC F16B 23/0007; B23B 51/0003; B25B 15/004

(57) ABSTRACT

The present invention provides a universal fastening system that joins or affixes two or more objects together. The universal fastening system comprises a driving tool having a first end and a second end connected through a cylindrical shaft, wherein one end is a tip end having a plurality of indentations, and the other end is a tool head for receiving a driving shaft which can be rotated by a hand-driven tool or motorized tool. The system also includes a fastener comprising a head part on which a plurality of protrusion or teeth are positioned to facilitate the engagement of the fastener with the driving tool at various angles. Furthermore, the fastener also includes a shank member centrally attached to the head of the fastener, having a plurality of threads enables the insertion of the fastener within the fastening channel when rotated by the driving tool.

### CROSS REFERENCE TO RELATED APPLICATION

This application claims priority to provisional Patent Application No. 63/140,246 filed Jan. 21, 2021, Application No. 63/152,962 filed on Feb. 24, 2021, Application No. 63/217,302 filed on Jul. 1, 2021, and Application No. 63/237,537 filed on Aug. 27, 2021, which application is herein incorporated by reference for all that it discloses.

### TECHNICAL FIELD

The present invention is related generally to universal fastening system assembly for the medical, construction industry, electrical, lightening, access control, fencing, balustrading, architecture, aerodynamics, robotics, automotive, infrastructure, agriculture, military, and other similar industries. More specifically, the present invention relates to a universal fastening system that can be used to join or hold materials together generally as a non-permanent joint.

### BACKGROUND ART

Conventionally available fastening tools like bolts, screws, etc., are the most common type of fastening tools employed in every type of industry and require a specific angle (most of the time 180 or 0 degrees) to operate. These devices are limited to a particular rotating angle, the angle at which the driver rotates these tools and facilitates their insertion in the fastening channels. The driver that rotates the bolt, screw, or any other fasteners, only engages with the drives or groove made on the tool when operating at a 180- or 0-degree angle. If it is operated at any angle other than specific, then the tip of the driver does not mesh or engage with drives of the fastening tool, and the tool will not be rotated.

Furthermore, these tools usually need more space for working. Sometimes, in areas where less space for working is available, for example, dental implants, machinery, automotive, and areas with tight spots, it becomes very difficult to rotate these tools and cannot be fixed properly. Also, improper rotation of these fastening tools causes the damage or destruction of the channel where fastening tools are inserted through rotational motion. Areas having tight spot prohibits the working of the driver and provide a significantly less angle of rotation.

Moreover, one of the significant drawbacks of the conventionally available fastening tools is that they get easily affected by atmospheric conditions, for example, rust or corrosion, which leads to failures in the fastening system. Preferably metal fasteners are expected to last and be the glue that holds structures and their components together. But when they come in direct contact with the atmospheric conditions (rain, moisture), the electrochemical reaction takes place and affects aesthetic features (the outer appearance of fastener design or, more specifically, the head of the metal fasteners) of the fastener. Due to which the drives or recesses present on the fastener's head are faded and damaged. Corrosion can cause a significant reduction in the strength of the fastener, for example, the metallic strength of the fastener, which could lead to a break or failure of fasteners.

Stripping of fasteners is one of these problems that play a significant role in the installation of fasteners. Stripping arises due to less friction, the surface area of engagement, and torque between the driving tool and the fastener's head. Continuous use of fasteners and their less and improper engagement with the driving tool are the main reasons for stripping issues among the fasteners. A stripped fastener is a fastener whose head is damaged so much that it is difficult to remove with a driving tool. Damaged or broken head of a fastener affects the structural integrity of the tool and degrade the functioning of fasteners. A fastener with a stripped head leads to skidding the driving tool over the fastener, and their damaged profile creates problems in installing fasteners and subsequently compromises the fastening complex. To eliminate the sliding of the driving tool over the fastener's head, one needs to increase the friction, surface area of engagement, and torque between them.

In a conventionally available fastening system, the fastener's inaccessibility and unreachability of drives is a significant issue in removing fasteners. In scenarios where the drives of the fasteners flushed within the surface or bodies of the object and drives are not available for rotation purposes, it is impossible to remove the fasteners. Hard-to-reach fasteners sometimes lead to the failure of the fastening system. In order to get access to these fasteners, one may tap or hit against the fastener and cut that part to loosen the fastener, use a reverse motorized action with a bur, or not have the ability at all to recover. These situations will affect the structural configurations of the fasteners and reduce their efficiency.

Therefore, there is a need for a universal fastening system that can be operated at various angles, requires less workspace, aesthetically closes the system from external environments, and removes the stripping, angulation, and slipping problems. Furthermore, a fastening system also needs to facilitate the easy removal and recovery of fasteners and components when their drives are not accessible and also provides a solution to the drawbacks and deficiencies associated with the prior art.

### SUMMARY

In an aspect of the present invention, a fastening system is provided. The fastening system comprises: a fastener includes a head comprising a plurality of protrusions, and a shank centrally attached with the head, wherein said shank further includes a plurality of male threads allowing the insertion of the fastener within a fastening channel; a driving tool comprising a first end and a second end connected through a cylindrical shaft, the first end is a tip end having a plurality of indentations that facilitate the engagement of driving tool with the plurality of protrusion on the fastener at different angles. The second end of the driving tool is a tool head for receiving a driving shaft rotatable by a hand-driven tool, a motorized tool, or a latch driver.

In another aspect, the shape of the indentation on the driving tool and the protrusion of the fastener includes but is not limited to V-shape, cycloidal shape, worm-gear shape, herringbone gear shape, and spur gear shape.

In another aspect, the shank and head of the fastener can be detachably attached with each other by means of a snap and fit mechanism, threaded mechanism, magnetic mechanism, or any other connecting mechanism. The fastener is selected from a group containing non-permanent fasteners, wherein non-permanent fasteners include but are not limited to screw fasteners, bolt fasteners, or any other threaded fasteners.

In another aspect, the tip end of the driving tool acts as a jammer when detached from the cylindrical shaft. The jammer is used where the head of the fastener is not accessible, such that the jammer is fixed with the fastener head and is kept accessible from the outside using a driver to tighten or loosen the fastener.

In another aspect, the head of the fasteners has a semi-spherical structure having protrusions or teeth positioned to allow the engagement of the driving tool at different angles. The fastener is selected from a group containing non-permanent fasteners, wherein non-permanent fasteners include but are not limited to screw fasteners, bolt fasteners, or any other threaded fasteners. The driving tool, when engaged, the clockwise and anticlockwise rotation of the driving tool causes the fastener to rotate accordingly.

In another aspect, the head of a fastener has a tapered shape comprising a plurality of protrusion or teeth that are positioned or arranged in a way to facilitate engagement of a driving tool with the fastener at different angles.

In another aspect, the fastener further comprises a support element that supports an object placed over the fastener. The object is being employed to cover the whole portion of the fastener. The support element comprises a receiving slot for receiving an extruded part of the object, causing the proper fixation of the object over the support element.

In another aspect, the fastener comprises a movable adaptor provided on the shank of the fastener. The adaptor has a hexagonal structure at the base and a semi-circular structure from the top. The adaptor is mechanically driven about the central axis of the fastener.

In another aspect, the fastener is mounted inside an object. The object comprises a recessed portion that receives the head of the fastener, and the head is held tightly by the driving tool. A covering member is non-permanently fixed to the object by means of a snap, friction, locking, cement, magnetic, glue, resin, or any other connecting mechanism.

In another aspect, the fastening system further comprises an anti-skidding element on top of the head of the fastener. The anti-skidding element is detachably attached with the head of the fastener.

In another aspect, the driving tool further comprises a protrusion member that extends centrally from the driving tip. The protrusion member gets engaged with a receiving slot located centrally on the fastener's head. The protrusion member can be hexagonal, V-shape, helical, cycloidal, bevel, S-shaped, wave-shaped, circular, square, rectangular, triangular, polygonal, and similar; or any tapered configurations thereof. The protrusion member can be located on the head of the fastener, and the receiving slow is on the driving tool.

In another aspect, the fastening system further comprises a jammer having a plurality of indentations that engages with a plurality of protrusion of the fastener at multiple angles.

In some aspects, the tip end of the driving tool is used as the jammer. The bottom portion of the jammer comprises a groove configured to receive a drive shaft.

In another aspect, the fastening system includes a fastener comprising an electronic chip that can be further used for Artificial Intelligence (AI) based applications.

In another aspect, the fastening system further comprises a nut with a circular body with a top and bottom surfaces. In addition, the nut includes a threaded hole that allows the rotation of the nut over the fastener.

In accordance with another aspect, the present invention provide a fastening system comprising a plurality of mechanical components such as drivers, fasteners, anti-skidding element, covering member, nut, bolt and the like are independent of shape size, may also include micro-versions and macro-versions suitable for specific applications. Depending upon the shape and size of each and every component of the universal fastening system are readily provided with a tool kit for retailing purposes

In another aspect, the fastening system comprises a lifter configured to cut the material circumferentially and drill the material centrally. The lifter is used to vertically or horizontally lift the solid material to a desired height or length. The lifter comprises a cylindrical body having a plurality of cutting blades provided at one end. The cylindrical body includes a bore extending axially for receiving material cut by the cutting blades. The lifter further comprises a drill bit.

In accordance with another aspect, there is provided a fastening system that includes one-piece or multi-piece configurations.

### BRIEF DESCRIPTION OF DRAWINGS

FIG. 1A illustrates a perspective view of a driving tool, in accordance with an embodiment of the present invention.

FIG. 1B illustrates an enlarged view of the bottom side of the driving tool in accordance with an embodiment of the present invention;

FIG. 2A illustrates a perspective view of the fastening tool, when the driver engages with a fastener at 0-degree, in accordance with an embodiment of the present invention;

FIG. 2B illustrates a side view of the fastening system, when the driver engages with a fastener at 0-degree, in accordance with an embodiment of the present invention;

FIG. 3 illustrates a side view of the fastening system, when the driver engages with a fastener at 45-degree, in accordance with an embodiment of the present invention;

FIG. 4 illustrates a side view of the fastening system, when the driver engages with a fastener at 90-degree, in accordance with an embodiment of the present invention;

FIG. 5 illustrates a side view of the fastening system, when the driver engages with a fastener at 135-degree, in accordance with an embodiment of the present invention;

FIG. 6 illustrates an alternate embodiment of the fastener, where the head of the fastener can be detached from the shank.

FIG. 7a illustrates a perspective view of the fastener, in accordance with an embodiment of the present invention;

FIG. 7b illustrates a perspective view of the fastener when an object is fixed on it, in accordance with an embodiment of the present invention;

FIG. 7c illustrates a cross-sectional view of the fastener when an object is fixed over it, in accordance with an embodiment of the present invention;

FIG. 8 illustrates a perspective view of the fastener having an adaptor, in accordance with an embodiment of the present invention; and

FIG. 9 illustrates a perspective view of the driving tool, in accordance with an alternate embodiment of the present invention;

FIG. 10a illustrates an exploded view of a fastening system used to engage the driving tool with the fastener at 90 degrees engagement, in accordance with an embodiment of the present invention;

FIG. 10b illustrates a cross-sectional view of the fastening system, when a fixing member is attached to an anti-skidding element, in accordance with an embodiment of the present invention;

FIG. 10c illustrates a cross-sectional view of the fastening system, when the fixing member is flushed within the first object, in accordance with an embodiment of the present invention;

FIG. 11a illustrates an exploded view of the fastening system used to engage the driving tool with the fastener at 90 degrees engagement, in accordance with an embodiment of the present invention;

FIG. 11b illustrates a cross-sectional view of the fastening system, in accordance with an embodiment of the present invention;

FIG. 12A illustrates an exploded view of a fastening system used to engage the driving tool with the fastener at 90 degrees engagement, in accordance with an exemplary embodiment of the present invention;

FIG. 12B illustrates a perspective view of an abutment, in accordance with an exemplary embodiment of the present invention;

FIG. 12C illustrates a perspective view of an abutment, in accordance with an alternate embodiment of the present invention;

FIG. 13 illustrates a cross-sectional view of the fastening system, in accordance with an exemplary embodiment of the present invention;

FIG. 14a illustrates the universal fastening system, when a covering member is detached from the dental crown, in accordance with exemplary embodiment of the present invention;

FIG. 14b illustrates a dental crown with a covering member in accordance with an alternate embodiment of the present invention;

FIG. 15 illustrates a perspective view of the driving tool disclosed in FIG. 12A, in accordance with an alternate embodiment of the present invention;

FIG. 16 illustrates an isometric view of the fastening system when the driving tool (disclosed in FIG. 15) engages with the fastener, in accordance with an embodiment of the present invention;

FIG. 17a illustrates a perspective view of a fastening system in accordance with an embodiment of the present invention;

FIG. 17b illustrates a front view of the fastening system when a driving tool engages with a fastener at a zero-degree angle in a straight-line approach in accordance with an embodiment of the present invention;

FIG. 17c illustrates a perspective view of the fastening system in accordance with an alternate embodiment of the present invention;

FIG. 17d illustrates a perspective view of the fastening system in accordance with an alternate embodiment of the present invention;

FIG. 17e illustrates a perspective view of the fastening system disclosing a driving tool and a fastener having micro-version suitable for specific applications in accordance with an alternate embodiment of the present invention;

FIG. 17f illustrates a perspective view of the fastening system disclosing a driving tool and a fastener having macro-version suitable for specific application in accordance with an alternate embodiment of the present invention;

FIG. 18a illustrates a perspective view of the fastening system in accordance with an embodiment of the present invention;

FIG. 18b illustrates a front view of the fastening system when the driving tool engages with the fastener at a zero-degree angle in accordance with an embodiment of the present invention;

FIG. 19 illustrates an exploded view of a fastener, in accordance with an embodiment of the present invention;

FIG. 20 illustrates a sectional view of the fastening system when a first object is joined to a second object in accordance with an embodiment of the present invention;

FIGS. 21A, 21B, and 21C illustrate a fastening cap that covers or hides the head of the fastener in accordance with an embodiment of the present invention;

FIG. 22A-22D illustrate a perspective view of the driving tip of the driving tool in accordance with alternate embodiments of the present invention;

FIG. 23a illustrates a perspective view of the fastening system when a non-threaded fastener is being employed for fastening purpose in accordance with an embodiment of the present invention;

FIG. 23b illustrates a perspective view of a fastener having deep threads or self-threading mechanism in accordance with an alternate embodiment of the present invention;

FIG. 23c illustrates a front view of a fastener having a thin head for 0-degree engagement in accordance with an alternate embodiment of the present invention;

FIG. 23d illustrates a cross-sectional view of a fastener (0-degree fastener) having an internal threaded channel for receiving another threaded fastener, in accordance with an alternate embodiment of the present invention;

FIG. 24a illustrates a perspective view of a fastening system in accordance with an embodiment of the present invention;

FIG. 24b illustrates a perspective view of a universal jammer in accordance with an embodiment of the present invention;

FIG. 25a illustrates a perspective view of a fastening system when the universal jammer is engaged with the fastener in accordance with an embodiment of the present invention;

FIG. 25b illustrates a perspective view of a fastening system when a handheld driver drives the universal jammer in accordance with an alternate embodiment of the present invention;

FIG. 26a illustrates a perspective view of a driving bit in accordance with an embodiment of the present invention.

FIG. 26b illustrates a perspective view of a fastening system when the driving tip of FIG. 26a engages with the fastener in accordance with an embodiment of the present invention;

FIG. 27a illustrates a perspective view of an anti-skidding element in accordance with an embodiment of the present invention;

FIG. 27b illustrates a perspective view of an anti-skidding element in accordance with an alternate embodiment of the present invention;

FIG. 27c illustrates a bottom perspective view of an anti-skidding element in accordance with an alternate embodiment of the present invention;

FIG. 28 illustrates a removal operation of the anti-skidding element with the help of a driving tool in accordance with an embodiment of the present invention;

FIG. 29 illustrates a front view of a fastener when engaged with a driving tool at a 45-degree angle in accordance with an alternate embodiment of the present invention;

FIG. 30 illustrates a front view of a fastener when engaged with a driving tool at a 135-degree angle in accordance with an alternate embodiment of the present invention;

FIG. 31 illustrates a perspective view of a universal nut and bolt assembly in accordance with an embodiment of the present invention;

FIG. 32 illustrates a driving and guide tool that engages with the universal nut in accordance with an alternative embodiment of the present invention;

FIG. 33 illustrates a perspective view of a universal lifter in accordance with an embodiment of the present invention;

FIG. 34a illustrates an engagement of the fastener with a material that is cut by the universal lifter disclosed in FIG. 33 of the present invention;

FIG. 34b illustrates an engagement of the fastener with a material in accordance with an alternate embodiment of the present invention;

FIG. 35a illustrates a perspective view of a handheld universal driver in accordance with an embodiment of the present invention; and

FIG. 35b illustrates the internal components of the handheld universal driver disclosed in FIG. 35a of the present invention.

### DETAILED DESCRIPTION OF THE PRESENT INVENTION

The present invention discloses a universal fastening system that joins or affixes two or more objects together, where a driver engages with a fastener at various angles.

In an embodiment of the present invention, there is provided a universal fastening system that joins or affixes two or more objects together the fastening system comprising a driving tool that engages with a fastener at various angles. The driving tool includes a first end and a second end connected through a cylindrical shaft, wherein the first end is a tip end having a plurality of indentations and the second end is a tool head for receiving a driving shaft that can be rotated by a hand-driven tool or a motorized tool. The fastener of the present invention includes a head having a plurality of protrusion or teeth, structured in a way that facilitates the engagement of the driving tool with the fastener at different angles. The fastener further includes a shank centrally attached with the head, wherein the shank comprises a plurality of male threads allowing the insertion of the fastener within a fastening channel or area where they are rotated by the driving tool.

The shape of the indentations and protrusion includes but is not limited to V-shape, cycloidal shape, worm-gear shape, herringbone gear shape, and spur gear shape. The head of the fasteners preferably has a semi-spherical structure on which the protrusions or teeth are positioned in a way so that the driving tool can be engaged at different angles. The shank and head of the fastener can be detachably attached with each other by means of a snap and fit mechanism, threaded mechanism, magnetic mechanism, or any other connecting mechanism.

The fastener is selected from a group containing non-permanent fasteners, wherein non-permanent fasteners include but are not limited to screw fasteners, bolt fasteners, or any other threaded fasteners.

The driving tool can be driven by the hand-driven tool, motorized tool, and a latch driver. When engaged, the clockwise and anticlockwise rotation of the driving tool causes the fastener to rotate accordingly. The tip end of the driving tool can also act as a jammer when detached from the cylindrical shaft. The jammer is used where the head of the fastener is not accessible, so the jammer is fixed with the fastener head, and it is kept accessible from the outside anytime using a driver to tight/lose the fastener. The jammer may include a receiving slot provided at the bottom surface, which receives a driving shaft that rotates the jammer in a clockwise and anti-clockwise direction.

According to another embodiment, the head of a fastener has a tapered shape comprising a plurality of protrusion or teeth that are positioned or arranged to facilitate engagement of a driving tool with the fastener at different angles. The fastener further includes a support element (also has a tapered structure) that supports an object placed over it. The object is being employed to cover the whole portion of the fastener. This aspect of the present invention is useful in areas where the physical appearance of the fastener is not necessarily visible. E.g., in dentistry, where a crown is placed over an abutment, the abutment can be replaced with the fastener of the present invention, and the top surface of the fastener can act as a support element for the crown. Further, the head having a plurality of protrusions can be driven in the implant by the aforementioned driving tool.

The support element may include a receiving slot where an extruded part of the object is received, causing the proper fixation of the object over the support element.

According to another embodiment of the present invention, the fastener includes a movable adaptor provided on the shank part of the fastener, wherein the adaptor has a hexagonal structure its base and semi-circular structure from its top. In alternate embodiments, the adaptor may have a different shape that is identical to the structure of receiving, where the base of the adaptor is inserted. The adaptor is mechanically driven about the central axis of the fastener. When the desired insertion of the fastener is achieved, and the hexagonal base of the adaptor is fixed in its corresponding receiving, the head of the fastener contacts the upper surface of the adaptor and prohibits its further movement. One of the primary use of this structure is dentistry, where the hexagonal hollow head of the implant can fix with the hexagonal base of the adaptor, and its further movement is restricted by the fastener's head that contacts the adaptor.

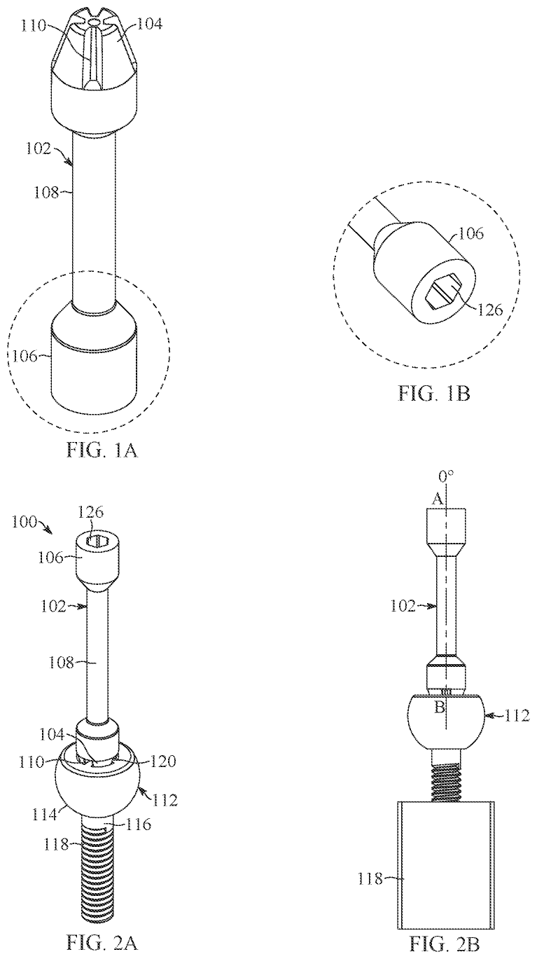

Referring now to FIG. 1A of the present invention illustrating the perspective view of driving tool 102. The driving tool 102 comprises a first end and a second end connected through a cylindrical shaft 108, wherein the first end is a tip end 104 having a plurality of indentations 110 and a second end is a tool head 106. The indentations 110 of the driving tool 102 can be of any shape, e.g., V-shape, cycloidal shape, worm-gear shape, herringbone gear shape, spur gear shape, etc. that enables the proper engagement of driving tool 102 with the fastener 112, 212, 312 and 412 shown in FIGS. 2, 3, 4 and 5 respectively. The driving tool 102 can be made of a rigid material like steel, iron, or a material having a coating of nickel or chromium that provides high strengthening or durability to the tool.

Further, FIG. 1B represents the enlarged view of the bottom side of the tool head 106. The tool head 106 of the driving tool 102 includes a hexagonal receiving or slot 126 where it receives a driving shaft that drives or rotates the driving tool 102 clockwise and anticlockwise direction, either manually or automatically. Apart from the hexagonal shape, the receiving slot 126 may include a tapered shaped, circular shape, rectangular shape, or any other shape that sufficiently fits the driving shaft that can be driven by a hand-driven tool, a motorized tool, and a latch driver.

Referring now to FIGS. 2A and 2B of the present invention illustrates a first embodiment of a fastening system 100 at 0-degree engagement (seen from the central axis A-B). There is shown a fastener 112 composed of low-medium carbon steel wire, brass, stainless steel, nickel alloy or aluminum, etc., that offers high strengthening and durability. The fastener 112 can be also be made of a material that is elastic, medical-grade, flexible with and/or without elasticity, rotatable, cost-efficient, easily manufacturable, plastically deformable, hygienic, sterile, tough, and/or lightweight. The fastener comprises a head 114 having a plurality of protrusion 120 positioned on the upper surface of the head 114, which are identical in shape to the corresponding indentations 110 of the driving tool 102. The protrusion 120 can be made of a material like a cast iron, aluminum alloy, brass, bronze that offers good mechanical properties. Moreover, the protrusion 120 can also be made of a rigid plastic material such as ABS, polycarbonate, PPSU, offering outstanding toughness during mechanical operations. At the 0-degree engagement, the indentations 110 of the driving tool 102 is completely meshed with the protrusion 120 of fastener 112, providing sufficient force to rotate the fastener 112 in the clockwise and anti-clockwise direction. Furthermore, the fastener 112 also comprises a shank 116 shown in FIG. 2A and FIG. 2B, attached with the head 114 of fastener 112. The shank 116 further includes a plurality of male threads 118 that engages with the female threads of fastening channel 122 or area (shown in FIG. 2B) where the fastener 112 is inserted due to rotation of driving tool 102. Moreover, the clockwise rotation of the driving tool 102 causes the fastener 112 to be inserted within the hollow casing of the fastening channel 122, and the anti-clockwise rotation of the driving tool results in the ejection or taking of the fastener 112 from the fastening channel 122.

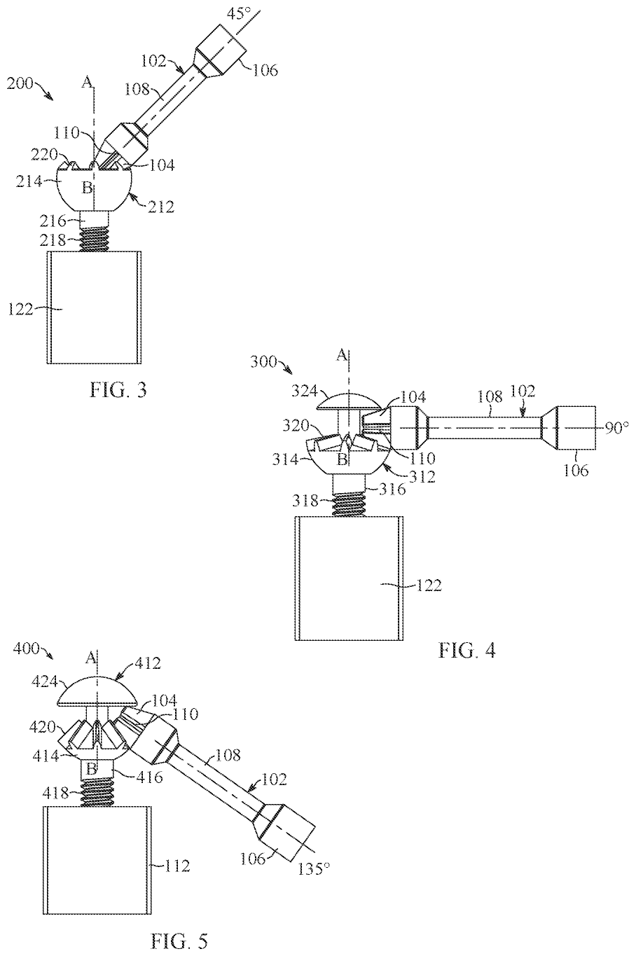

Referring now to FIG. 3 of the present invention illustrating a second embodiment of the universal fastening system 200 at 45-degree engagement (when seen from the central axis A-B). There is shown a fastener 212 comprising a head 214 having a plurality of protrusion 220, which are identical in shape to the corresponding indentations 110 of the driving tool 102. At the 45-degree engagement, the indentations 110 of the driving tool 102 is partially meshed with the protrusion 220 of fastener 212, providing sufficient force to rotate the fastener 212 in clockwise and anti-clockwise directions. Further, there is shown a shank 216 centrally attached with the head 214 of the fastener 212. The shank 216 includes a plurality of male threads 218 that engages with the female threads of fastening channel 122 when the fastener 212 is rotated by the driving tool 102.

Referring now to FIG. 4 of the present invention illustrating a third embodiment of the universal fastening system 300 at 90-degree engagement (when seen from the central axis A-B). There is shown a fastener 312 comprising a head 314 having a plurality of protrusion 320, which are identical in shape to the corresponding indentations 110 of the driving tool 102. At 45-degree engagement, the indentations 110 of the driving tool 102 is partially meshed with the protrusion 320 of fastener 312, providing sufficient force to rotate the fastener 312 in the clockwise and anti-clockwise direction. Further, there is shown a shank 316 centrally attached with the head 314 of the fastener 312. The shank 316 includes a plurality of male threads 318 that engages with the female threads of fastening channel 122 when the fastener 312 is rotated by the driving tool 102. Furthermore, there is also provided an anti-skidding element 324, which facilitates the steady rotation of the driving tool 102 over the fastener 312 without causing any damage.

Referring now to FIG. 5 of the present invention illustrating a fourth embodiment of the universal fastening system 400 at 135-degree engagement (when seen from the central axis A-B). There is shown a fastener 412 comprising a head 414 having a plurality of protrusion 420, which are identical in shape to the corresponding indentations 110 of the driving tool 102. At 45-degree engagement, the indentations 110 of the driving tool 102 is partially meshed with the protrusion 420 of fastener 412, providing sufficient force to rotate the fastener 412 in the clockwise and anti-clockwise direction. Further, there is shown a shank 416 centrally attached with the head 414 of the fastener 412. The shank 416 includes a plurality of male threads 418 that engages with the female threads of fastening channel 422 when the fastener 212 is rotated by the driving tool 102. An anti-skidding element 424 is also provided on the top of the head 414 that facilitates the steady rotation of the driving tool 102 over the fastener 412 without causing any damage. According to an alternate embodiment, for smooth engagement between the fastener 412 and the driving tool 102, it is preferable, the height of the anti-skidding element 424 should be the same as the height of the fastener's head 414.

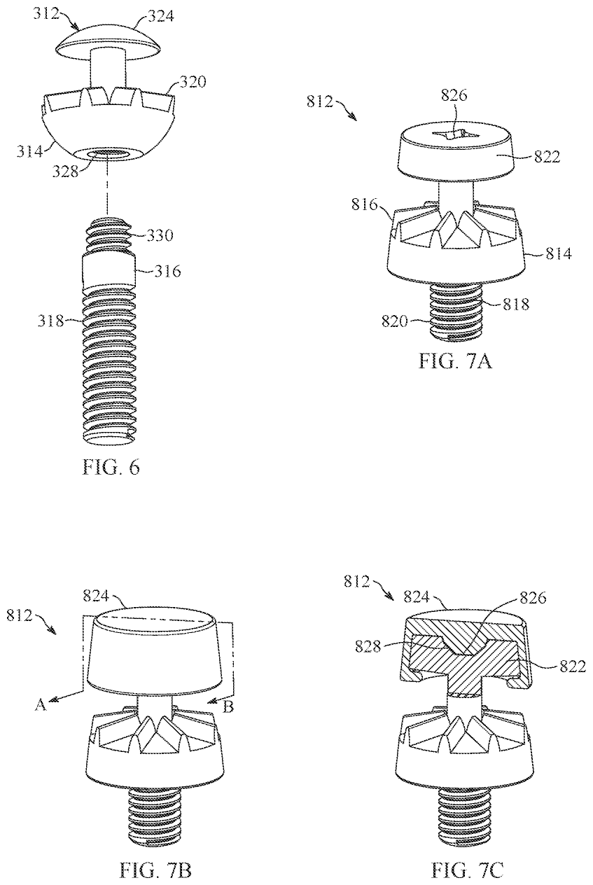

Referring now to FIG. 6, illustrating an alternate embodiment of the present invention, showing the detachable connection between the head 314 and shank 316 of the fastener 312, which is being employed for 90-degree engagement. The fastener 312 includes a head 314 on which a plurality of protrusions 320 are positioned and a circular opening or slot 328 positioned on the bottom surface of head 314. The circular opening or slot 328 receives the one end 330 of the shank 316 through a connecting mechanism. More particularly, the circular opening or slot 328 has female threads engaged with the male threads 318 of the shank 316 and causing the attachment or detachment of head 314 with respect to the shank 316. However, the connecting mechanism between the head 314 and a shank 316 includes but is not limited to threaded connections, snap and fit mechanism, magnetic mechanism. Also, there is shown an anti-skidding element 324 that prohibits the skidding effect. However, this figure is not limited to only a 90-degree engagement fastener, it may also include other fasteners that support other engagement angles, i.e., 0-degree, 45-degree, and 135-degree. For example, we can also employ other fasteners 112, 212, and 412 having detachable connections when other engagement angles are required. The detachable connection among the different types of the head (114, 214, 314, 414) and their shank (116, 216, 316, 416) enables the universal fastening system to be operated at multiple angles.

Referring now to FIG. 7a of the present invention illustrates a perspective view of the fastener 812 according to the sixth embodiment of the present invention. This embodiment of the present invention is helpful in areas or fields where it is necessary to hide the structure of fastener 812, for example, in the automobile industry or in dentistry where a crown is placed over an abutment. As shown in the figure, the fastener 812 comprises a tapered shape head 814 having a plurality of protrusions 816 that engages with indentations 110 of driving tool 102 (shown in FIG. 1A) and a shank 818 attached with the head 814 of fastener 812. The shank also includes a plurality of male threads 820 are configured to engage with the female threads of the fastening channel 122 (shown in FIG. 2). In addition, the fastener 812 further includes a support element 822 mounted at the top of the head 814, wherein the support element 822 also has a tapered shape and has a draft angle of 6˜12 degrees with respect to the head 814 of the fastener 812. The draft angle between the support element 822 and head 814 should be in the suitable range because, in this range, an object 824 (shown in FIGS. 7b and 7c) is easily fixed over the support element 822 and covers the fastener 812. Also, the tapered shape of both support element 822 and head 814 of the fastener 812 enables the object 824 to easily snap on and snap off over the fastener 812. In some embodiments, the fastener 812 may include a receiving slot 826 where an extruded part 828 of the object 824 is received, causing the proper fixation of object 824 over the support element 822, as shown in FIG. 7c. The receiving slot 826 is provided at the top surface of the support element 822.

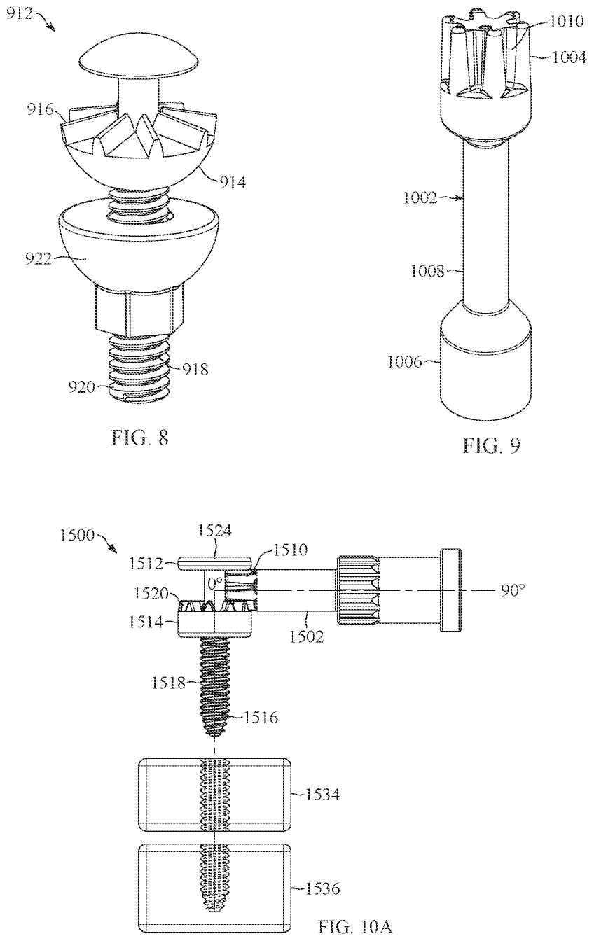

Referring now to FIG. 8 of the present invention illustrates the perspective of the fastener, according to the eighth embodiment of the present invention. As shown in the figure, the fastener 912 comprises a head 914 having a plurality of protrusions 916 that engages with indentations 110 of driving tool 102 (shown in FIG. 1A) and a shank 918 attached with the head 914 of fastener 912. The shank 918 also includes a plurality of male threads 920 that are configured to engage with the female threads of the fastening channel 122 (shown in FIG. 2A).

The fastener 912 further includes a movable adaptor 922 provided on the shank 918 part of the fastener 912, wherein the adaptor 922 has a hexagonal structure (or can be of any shape identical to the receiving member) on its base and semi-circular structure on its top. The adaptor 922 also has a respective thread engage with the threads of shank 918 and is free to move in an upward and downward direction about the central axis of the fastener 912. In application, when the protrusion 916 of fastener engaged with the indentation 110 of driving tool 102 (shown in FIG. 1A) and rotation of fastener 912 begins, the adaptor 922 moves in a downward direction, and its hexagonal base is fixed within the corresponding receiving. At this stage, head 914 of the fastener 912 contacts the upper surface of the adaptor 922 and prohibits or locks the further movement of adaptor 922.

Referring now to FIG. 9 illustrating the perspective view of the driving tool 1002, in accordance with an alternate embodiment of the present invention. In this embodiment, the tip end 1004 of the driving tool 1002 has a cylindrical structure having a plurality of indentations 1010, the cylindrical structure of driving tool 1002 enables the effective engagement of the driving tool 1002 with the fasteners (112, 212, 312, and 412) at different angles.

In another embodiment, the present invention discloses a fastening system that joins or affixes two or more objects together, where a driving tool is engaged with a fastener at various angles.

In an embodiment of the present invention, the universal fastening system includes a fastener mounted inside the first object. The first object hides the exposed surface of the fastening head and may have a round, square, rectangular, polyhedral shape depending on the user's convenience and the application area where the fastening system is employed. For example, the first object can be a dental crown used in dental implants or can be a screw cover for hiding a screw head used in different industrial sectors. The first object provides a simple yet effective solution to conceal and protect exposed fastener's head and is used across multiple industries such as automotive and healthcare, however, often used in furniture, infrastructure, and building applications too. The first object may include a recessed portion that receives the fastener head, and the fastener head is being held tightly by the driving tool in the air. A covering member is non-permanently fixed to the first object by means of a snap, friction, locking, or suitable mechanism, magnetic, cement, glue, resin, or any other connecting mechanism. The covering member may have a similar color as the color of the first object and also have similar dimensions as the recessed portion of the first object. The covering member supports the fastener's head and prohibits further rotation as well as loosening of the fastener head with respect to the shank by covering the recessed portion of the first object.

In another embodiment of the present invention, a universal fastening system is provided that joins a first object and a second object together. The fastening system comprises a fastener having a head that is flushed within the surface of the first object in such a way that a plurality of protrusion positioned onto the head of the fastener remains outside of the first object. The fastener includes a shank centrally attached with the head, wherein the shank includes a plurality of male threads allowing the insertion of the fastener between the first object and the second object. The fastener further includes an anti-skidding element upon which a fixing member is mounted, covering the top portion of the fastener. The fixing member can be attached to the anti-skidding element by means of a snap and fit mechanism, threaded mechanism, magnetic mechanism, or any other connecting mechanism. The fixing member may include a flexible support made of silicon or any other soft material that allows the proper fixation of the fixing member according to the contour of the anti-skidding element.

According to another embodiment of the present invention, the anti-skidding element of the fastener can be detachably attached with the head of the fastener. As per this aspect, the fastening system of the present invention also includes a fixing member, which encloses the head of the fastener and protects it from outer atmospheric conditions. The fixing member can be attached to the first object by means of a snap and fit mechanism, threaded mechanism, magnetic mechanism, or any other connecting mechanism

According to another embodiment, the present invention provides a universal driving tool configured to join or affix two or more together. The driving tool includes a first end and a second end connected through a cylindrical shaft, wherein the first end is a tip end having a plurality of indentations and the second end is a tool head. The driving tool is configured to rotate clockwise and in the anti-clockwise direction, and its rotational motion can be controlled either manually or automatically. In one embodiment, especially in case of dental crown and abutment, the tip end of the driving tool provides stability during the fastening operation. The tip end stops the swivel movement of the driving tool when engaged with a fastener at a particular angle. In use, when a first object having a recessed portion is attached with a second object using the driving tool, the circumferential part of the tip end contacts with the boundary of the recessed portion of the first object and thereby prohibits the swivel movement of the driving tool with respect to the fastener.

The shape of the indentations and protrusion includes but is not limited to V-shape, cycloidal shape, worm-gear shape, herringbone gear shape, and spur gear shape. The head of the fasteners preferably has a semi-spherical structure on which the protrusions or teeth are positioned in a way so that the driving tool can be engaged at different angles. The shank and head of the fastener can be detachably attached with each other by means of a snap and fit mechanism, threaded mechanism, magnetic mechanism, or any other connecting mechanism.

The fastener is selected from a group containing non-permanent fasteners, wherein non-permanent fasteners include but are not limited to screw fasteners, bolt fasteners, or any other threaded fasteners.

The driving tool can be driven by the hand-driven tool, motorized tool, and a latch driver. When engaged, the clockwise and anticlockwise rotation of the driving tool causes the fastener to rotate accordingly. Based on the geometric coupling configurations of fasteners and driving tools, an engagement angle from 0 to 180 degrees is achieved.

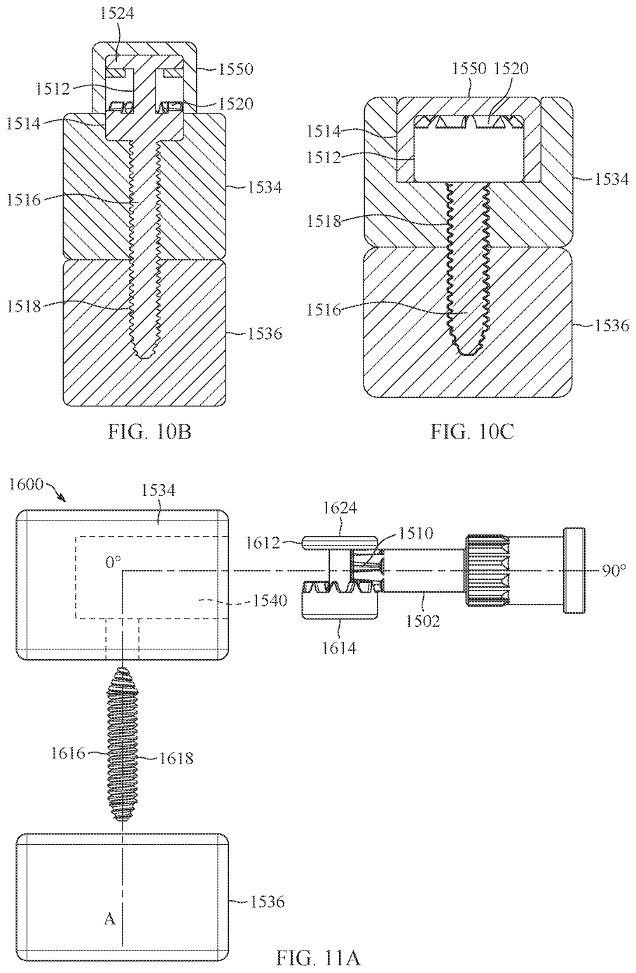

Referring now to FIG. 10A of the present invention illustrating a fastening system 1500 in accordance with the first embodiment, when a driving tool 1502 engages with a fastener 1512 at a 90-degree angle, in order to join a first object 1534 and a second object 1536 from the top surface. There is shown a fastener 1512 composed of solid materials such carbon steel, titanium, peek, brass, stainless steel, gold silver, bronze, any alloy, aluminum, etc., that offer high strengthening and durability. The fastener 1512 can be also be made of a material that is elastic, medical-grade, flexible with and/or without elasticity, rotatable, cost-efficient, easily manufacturable, plastically deformable, hygienic, sterile, tough, and/or lightweight. The fastener 1512 comprising a head 1514 has a plurality of protrusion 1520 positioned on the upper surface of the head 1514 and is structured to facilitate effective engagement with a plurality of indentations 1510 provided on one end of a driving tool 1502. The driving tool 1502 can be made of any rigid material like steel, iron, titanium, brass, gold, silver, or have a coating of nickel or chromium or similar material that provides high strengthening or durability to the tool. The driving tool 1502 can also be configured to rotate manually or automatically. For effective engagement, the plurality of protrusion 1520 should have a matching shape, pitch value, and dimensions as the corresponding plurality of indentations 1510 of the driving tool 1502. The engagement between the driving tool 1502 and the fastener 1512 enables the transmission of the rotary motion of the driving tool 1502 to the head 1514 of the fastener 1512, thereby enabling the clockwise and anti-clockwise rotation of the fastener.

The plurality of protrusions 1520 and the plurality of indentations 1510 of the fastening system 1500 may have a unique structure. The structure may include but is not limited to V-shape, cycloidal shape, worm-gear shape, herringbone gear shape, spur gear shape, or any other geometric configuration enabling the proper engagement of driving tool 1502 with the fastener 1512. The plurality of indentations 1510 of the driving tool 1502 and the plurality of protrusion 1520 of the fastener 1512 are perfectly engaged in a way that driving tool 1502 can easily carry and hold the fastener 1512 in the air by the force of friction. The fastener 1512 can also be held by the driving tool 1502 by other connecting mechanisms, such as magnetic and snap-fit connections. The plurality of protrusion 1520 and the plurality of indentations 1510 can be made of any solid materials like a cast iron, aluminum alloy, brass, bronze that offers good mechanical properties during the operation of the fastening system 1500. Moreover, the protrusion 1520 and indentations 1510 can also be made of a rigid plastic material such as ABS, polycarbonate, nylon, peek, or PPSU offering outstanding toughness during mechanical operations.

The fastener 1512 further includes an anti-skidding element 1524 mounted on the top of the head 1514 that facilitates the steady rotation of the driving tool 1502 over the fastener 1512. The anti-skidding element 1524 prohibits and reduces the skidding motion between the surfaces of the plurality of protrusions 1520 and indentations 1510. The anti-skidding element 1524 may be made of a material, such as cast iron, brass, plastic, and wood offering high resistance to skidding motion and firmly holding the driving tool 1502. Furthermore, the fastener 1512 also comprises a shank 1516 shown in FIG. 10a centrally attached with the head 1514 of fastener 1512. The shank 1516 further includes a plurality of male threads 1518 that engages with the female threads of the first object 1534 and the second object 1536, where the fastener 1512 is inserted due to rotation of driving tool 1502. Moreover, the clockwise rotation of the driving tool 1502 causes the fastener 1512 to be inserted within the hollow casing of the first object 1534 and second object 1536, and the anti-clockwise rotation of the driving tool results in the ejection or removal of the fastener 1512 from the first object 1534 and the second object 1536.

Referring now to FIG. 10b of the present invention, illustrating a cross-sectional view of the fastening system 1500, when a fixing member 1550 is attached to the anti-skidding element 1524. There is shown a fastener 1512 includes a head 1514 whose upper surface is flushed within the surface of the first object 1534, when the first object 1534 is affixed with second object 1536. The head 1514 of the fastener 1512 coincides with the first object 1534, in such a way, a plurality of protrusion 1520 positioned onto the head 1514 of the fastener 1512 remains outside of the first object 1534. This arrangement of the fastening system 1500 is useful in areas where a lower height of fastener 1512 is needed, and also it protects the head 1514 from being exposed under extreme environmental conditions, for example, rain, temperature, moisture. Also, one of the significant advantages of using this arrangement of the fastening system 100, it provides more stability to the fastener 1512 when it is fixed in the first object 1534.

The fixing member 1550 illustrated in FIG. 10b covers or encloses the anti-skidding element 1524 and plurality of protrusion 1520 positioned on the fastener's head 1514. The fixing member 1550 can be attached to the anti-skidding element 1524 by means of a snap and fit mechanism, threaded mechanism, glue, cement, resin, magnetic mechanism, or any other connecting mechanism. The fixing member 1550 can be made of any flexible material (plastic, fiber, glass, wood, rubber, ceramic, etc.) and may include a plurality of shapes depending on the user's convenience or application area where the present fastening system 1500 is being employed. The fixing member 1550 also exhibits high resistive features to extreme environmental conditions and protects the upper portion of fastener 1512 from the conditions that affect the physical and mechanical properties of the fastener 1512. The fixing member 1550 may include a flexible support made of silicon or any other soft material that allows the proper fixation of the fixing member 1550 according to the contour of anti-skidding element 1524.

In some embodiments, the plurality of protrusions 1520 of the fastener 1512 is kept inside the first object 1534, as shown in FIG. 10c, so that the fixing member 1550 is set flush with the top part of the first object 1534 after the removal of the anti-skidding element 1524. The anti-skidding element 1524 can be easily removed from the head 1514 of the fastener 1512 by snapping it using a tool or can be dismantled through threaded connection, friction, magnetic connection, or any other type of connection. The fixing member 1550 can be made of a material that exhibits high resistive features to extreme environmental conditions and protects the upper portion of fastener 1512 from the conditions that affect the physical and mechanical properties of the fastener 1512.

Referring now to FIG. 11a of the present invention, illustrating an exploded view of the fastening system 1600, in accordance with the second embodiment, when the driving tool 1502 engages with the fastener 1612 at a 90-degree angle. The fastening system 1600 includes the first object 1534 and the second object 1536 joined together using the driving tool 1502 and the fastener 1612 mentioned in FIG. 10a. The first object 1534 and the second object 1536 are connected using a shank 1616, having a plurality of male threads 1618. The fastener 1612 is being attached to the first object 1534 from sideways. In this embodiment, the head 1614 of the fastener 1612 can be detachably attached with the shank 1616 of the fastener 1612 through a connecting mechanism, allowing the compatibility of the fastening system 1600 being employed for multiple angle engagement. For example, in scenarios where a 45-degree engagement angle is needed, the fastener's head 1614 with protrusions suitable for 45-degree engagement can be fixed to the shank 1616 of the fastener. Similarly, different engagement angles, for example, 0-degree and 135-degree, can be achieved using the present fastening system 1600. Furthermore, as different types of the fastener's head 1514 have different structural configurations for various angles, the fastener's head 1514 can be attached easily with the shank 1516, leading to ease the fastening process. Furthermore, the shank 1616 and head 1614 of the fastener 1612 can be detachably attached with each other by means of a snap and fit mechanism, threaded mechanism, magnetic mechanism, glue, cement, resin, or any other connecting mechanism.

The first object 1534 shown in FIG. 11a, includes a recessed portion 1540 that receives the fastener's head 1514 that is tightly held by the driving tool 1502 by the force of friction. The recessed portion 1540 can be provided on any side of the first object 1534, depending upon the requirement of the fastening application. This enables the users to perform the fastening operation from a variety of engagement angles and directions. The head 1614 of the fastener 1612 can also be held by the driving tool 1502 by other connecting mechanisms, such as magnetic and snap-fit connections. Moreover, the anti-skidding element 1624 mounted on the top of the fastener's head 1614 also supports the driving tool 1502 in holding the head 1614 of the fastener 1612 in the air. The recessed portion 1540 may have sufficient space for the fastener's head 1614, allowing the head 1614 of the fastener 1612 to be perfectly aligned with the axis of which the shank 1616 is inserted within the first object 1534. The recessed portion 1540 that receives the fastener's head 1614 can be pre-set and may be of any geometric configuration.

Referring now to FIG. 11b of the present invention, illustrating a cross-sectional view of the fastening system 1600 described in FIG. 11a, when the first object 1534 is joined with the second object 1536. There is shown a covering member 1528 that covers the recessed portion 1540 of the first object 1534. The main objective of using the covering member 1528 in the fastening system 1600 is to protect the fastener 1612 from being exposed in the outer atmospheric conditions and to restore the aesthetic features of the first object 1534. Atmospheric conditions, such as heat, temperature, moisture, bodily fluids, rain, and wind, affect the outer appearance of fastener 1612 and get easily corroded by the electrochemical reaction when not covered. These conditions lead to the failures of the fastening system and destroy the structural integrity of the fastener 1612.

The covering member 1528 is non-permanently fixed to the first object 1534 by means of a snap and fit mechanism, friction, magnetic mechanism, cement, glue, resin, or any other connecting mechanism. The covering member 1528 may be of the same material as the first object 1534 or second object 1536 or any additional object to be joint and may have a similar or same color as the first object 1534. Also, the covering member 1528 may have similar dimensions as recessed portion 1540 of the first object 1534. The covering member 1528 can be of any solid material that exhibits good mechanical and chemical properties that are not affected by outer-atmospheric conditions. A resin composite material can also be used for covering member 1528 in order to protect the fastener 1612. When attached, the inner surface of the covering member 1528 contacts with the fastener's head 1614 and maintains the stability of the fastener's head 1614 at a fixed position. The contact may be lateral, vertical, horizontal, or any other arrangement to achieve such an objective. The covering member 1528 prohibits further rotation as well as the loosening of the fastener's head 1614 with respect to the shank by covering some or all of the accessible area of the recessed portion 1540 of the first object 1534. When the covering member 1528 is attached, the empty space of the recessed portion 1540, which is not accessible, can be left empty or filled with flexible, soft, or flowable materials such as cotton, cement, glue, Teflon, resin, light-cured materials, or similar prior to placement of the covering member 1628.

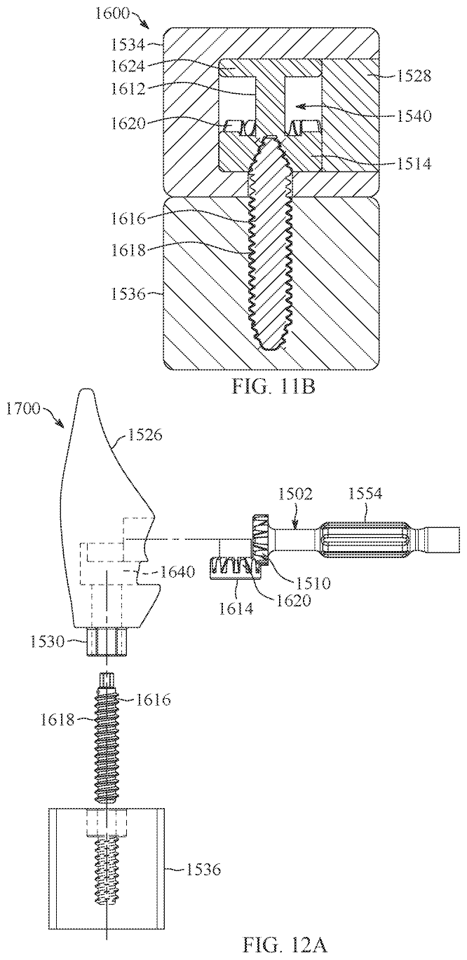

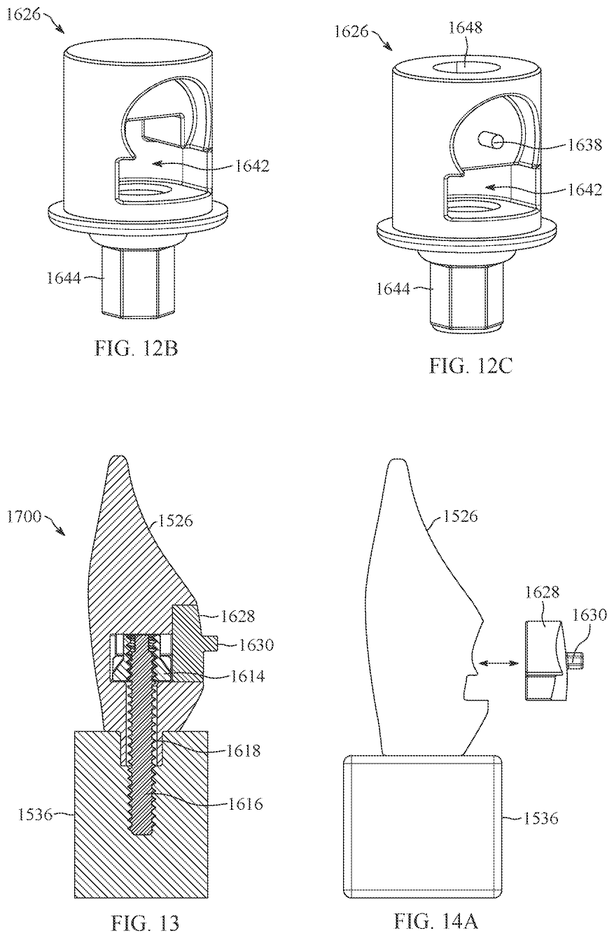

Referring now to FIG. 12A of the present invention, illustrating an exploded view of the fastening system 1700, in accordance with an exemplary embodiment, when the driving tool 1502 engages with the head 1614 of the fastener 1612 at a 90-degree angle. In the preferred embodiment, the provided fastener 1612 does not have any anti-skidding element. However, it may include the anti-skidding element in alternate embodiments depending upon the requirement of the application. The fastening system 1700 of the present invention applies to any industry where alignment between the driving tool 1502 and the fastener 1612 is difficult and needs frequent design modifications in the arrangement to fasten two or more objects together. In this embodiment, the first object 1534 (shown in FIG. 11a), is replaced by a prosthetic element used in the medial application, where a limited range of engagement angle is present. For example, in dentistry, the prosthetic element is a dental crown 1526 is used in dental implants. A dental crown 1526 is a type of dental restoration that completely caps or encircles a tooth or dental implant. The dental crown 1526 can be made out of metals, porcelain, resin, and ceramics that provide a natural look. Various configurations of the dental crown 1526 and the fastener 1612, which exist in the digital library, can be employed for the purpose of the invention. In healthcare, alternative embodiments can be applied to many orthopedics and prosthetic procedures, such as stabilizing broken bones, joint replacements, or anchoring for additional appliances or procedures.

The fastening system 1700 includes a dental crown 1526 and a second object 1536 joined together via the driving tool 1502 and the fastener 1612 illustrated in FIG. 12a. The dental crown 1526 includes a hexagonal protrusion 1530 provided at the bottom surface of the dental crown 1526 that is going to be fixed in the corresponding receiving (not shown) of the second object 1536 when joined by the shank 1616. The hexagonal protrusion 1530 also provides an anti-rotational feature and prohibits the rotation of the dental crown 1526 with respect to the second object 1536. The dental crown 1526 and the second object 1536 are connected using a shank 1616 of the fastener 1612. The fastener 1612 is being attached to the dental crown 1526 from sideways. In one embodiment, the second object can be an implant having internal threads, wherein said internal threads are configured to receive the shank 1616 of the fastener 1612. The implant can act as an anchor for the dental crown 1526 and interface with the bone of jaw or skull.

The dental crown 1526 shown in FIG. 12A includes a recessed portion 1640 that receives the fastener's head 1614, which is tightly held by the driving tool 1502 by the force of friction. The force of friction arises when the plurality of indentations 1510 meshes with the plurality of protrusion 1620 of the fastener 1612. The head 1614 of the fastener 1612 can also be held by the driving tool 1502 by other connecting mechanisms, for example, magnetic connection, cement, resin, and snap-fit connection. Depending on the requirement of the fastening application, the recessed portion 1640 can be present anywhere on the dental crown 1526. The dental crown 1526 with varying positioning of the recessed portion 1640 enables the user to fasten the prosthetic with the second object 1536 at different engagement angles and directions. For example, in one embodiment, the dental crown 1526 can be attached or accessed from the tongue side of the patient, or in alternate embodiments, it can be accessed from the cheek side of the patient. Therefore, it can be used with multiunit abutments, scan bodies, ti-based, and angled abutments as well. The recessed portion 1640 of the dental crown 1526 may have sufficient space for the fastener's head 1614, allowing the head 1614 of the fastener 1612 to be perfectly aligned with the central axis of the shank 1616 is inserted within the dental crown 1526. The shank 1616 of the fastener 1612 facilitates the rapid seating of the prosthetic on the user's gum and also acts as a guide to the dental crown 1526.

The arrangement enables the proper fixation of the dental crown 1526 over the second object 1536. Also, in the present fastening system 1700, micro-movements are reduced between the dental crown 1526 and the second object 1536 junction that arises due to the unbalanced forces acting on the dental crown 1526. These forces occur when the dental crown 1526 is not ideally positioned on the second object 1536. The aligned connection and seating force between the dental crown 1526 and the second object 1536 reduces the formation of micro gaps, micro-pumping and micro-movements, and screw loosening at recommended torque capacity per application. Moreover, the present arrangement preserves the occlusal and buccal surfaces and porcelain bulk for aesthetics and strengthening. Furthermore, the recessed portion 1640 provides an access, facilitating the easy removal of the head 1614 from the dental crown complex.

Referring now to FIG. 12B of the present invention, illustrating a perspective view of an abutment 1626 in accordance with an exemplary embodiment of the invention. In this embodiment, the dental crown 1526 is replaced by the abutment 1626, having a recessed portion 1642 of any shape configured to receive a head 1614 of matching shape, of fastener 1612 (illustrated in FIG. 12A). In some embodiments, the recessed portion 1642 may have a square, round, rectangular, and any other shape that eases the insertion of the fastener's head. The shape of the recessed portion 1642 provides the sufficient space to the fastener's head 1614, allowing the head 1614 of the fastener 1612 to be perfectly aligned with the central axis of the shank 1616. The abutment 1626 is a type of metal connector that connects the dental crown with the second object 1536 (illustrated in FIG. 12A). The abutment 1626 is configured to securely hold the dental crown in place with the second object 1536 via a connecting mechanism. In this case, the second object 1536 is a gingiva consisting of an implant and surrounding mucosal tissue.

The dental crown can be temporarily or permanently attached or stacked to the abutment 1626 by means of a snap and fit mechanism, friction, cement, glue, porcelain, resin magnetic mechanism, or any other connecting mechanism. In one embodiment, the abutment 1626 is made of titanium, peek, gold, stainless steel, zirconia, or polyether ether ketone material. Furthermore, the abutment 1626 also includes a hex-shaped base 1644 made of any metallic or non-metallic material and is received in the corresponding receiving of the second object 1536. In some embodiment, the abutment 1626 may include a base having circular, square, rectangular, triangular, and polygonal structures as well. In addition, the abutment 1626 of the present invention includes a specific structure that enables the fixation of the abutment 1626 with any type of existing implant system, which has been widely used in the dental and medical industry. The abutment 1626 can be connected to any type of existing implant system by matching the base of the abutment with the corresponding connections of the implant system. Furthermore, the shank 1616 can also be engaged with threads of the second object 1536, such as different types of the implant, to make the present fastening system universally applicable.

Referring now to FIG. 12C of the present invention illustrating a perspective view of an abutment 1626 in accordance with an alternate embodiment of the invention. In this embodiment, the abutment 1626 may include a cylindrical protrusion 1638 provided on the inner wall of the abutment. During the fastening operation, the cylindrical protrusion 1638 engages with the corresponding cavity 1646 of the driving tool 1502 (illustrated in FIG. 15). The primary purpose of the cylindrical protrusion 1638 is to improve the stability between the fastening components and also prohibit the lateral and swivel movement of the driving tool 1502 during operation. In this embodiment, the abutment 1626 also includes a thorough hole 1648 that can be further used for secondary purposes.

Referring now to FIG. 13 of the present invention, illustrating a cross-sectional view of the fastening system 1700 described in FIG. 12A, when the dental crown 1526 is joined with the second object 1536. There is shown a covering member 1628 that covers the recessed portion 1640 of the dental crown 1526. The covering member 1628 may include a structure corresponding to the recessed portion 1640 of the dental crown 1526. It can be accessed from a variety of angles and directions depending upon the positioning of the recessed portion 1640. The main objective of using the covering member 1628 in the fastening system 1700 is to protect the fastener 1612 from being exposed in the outer atmospheric conditions and restore the aesthetic features of dental crown 1526. Atmospheric conditions such as heat, temperature, moisture, rain, wind that affect the outer appearance of fastener 1612 when not covered. These conditions lead to the failures of the fastening system and destroy the structural integrity of the fastener 1612. The second significant advantage of employing the covering member 1628 onto the recessed portion 1640 of the dental crown 1526 is to maintain the aesthetic feature of the first body, in this embodiment as the dental crown, and improved emergence profile of the dental crown 1526 is achieved. When the covering member 1628 is attached, the empty area of the recessed portion 1640 which is not accessible can be left empty or filled with flexible, soft, or flowable materials such as cotton, cement, glue, Teflon, resin, light-cured materials, or similar prior to placement of the covering member 1628.

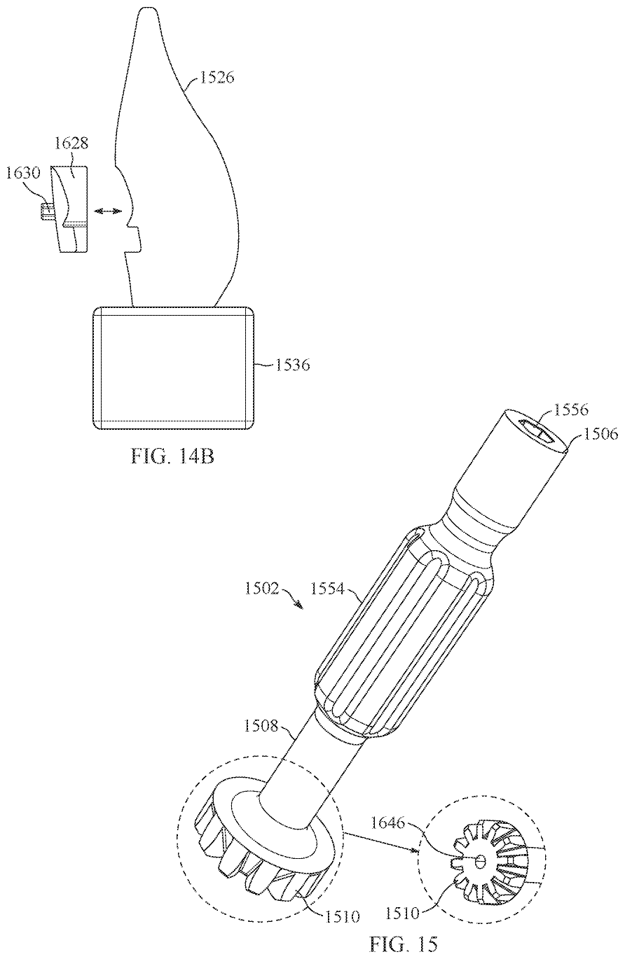

FIG. 14a illustrates the detachable connection between the recessed portion 1640 of the dental crown 1526 and covering member 1628. The covering member 1628 is detachably attached to the dental crown 1526 by means of a snap and fit mechanism, friction, cement, glue, resin magnetic mechanism, or any other connecting mechanism. The mechanism allows the rapid fixation and removal of the dental crown 1526 by attaching or detaching the covering member 1628 with the dental crown 1526, depending on the user's requirement. The covering member 1628 may be of the same material and color as the dental crown 1526 or any other materials or color. Also, the covering member 1628 may have similar dimensions and configurations as recessed portion 1640 of the dental crown 1526. When attached, the inner surface of the covering member 1628 contacts with the fastener's head 1614 and maintains the stability of the fastener's head 1614 at a fixed position. The covering member 1628 prohibits further rotation as well as the loosening of the fastener's head 1614 with respect to the shank by covering the recessed portion 1540 of the dental crown 1526. In some embodiments, the covering member 1628 may include a hex-shaped knob 1630 configured to receive by a slot 1556 of the driving tool 1502 shown in FIG. 15 of the present invention for removing or ejection purposes.

Referring now to FIG. 14b of the present invention illustrates a dental crown 1526 with a covering member 1628 in accordance with an alternate embodiment of the present invention. In this embodiment, the dental crown 1526 may include a recessed portion provided on its other side, and the covering member 1628 is attached to it by means of a connecting mechanism discussed in FIG. 14a of the present invention.

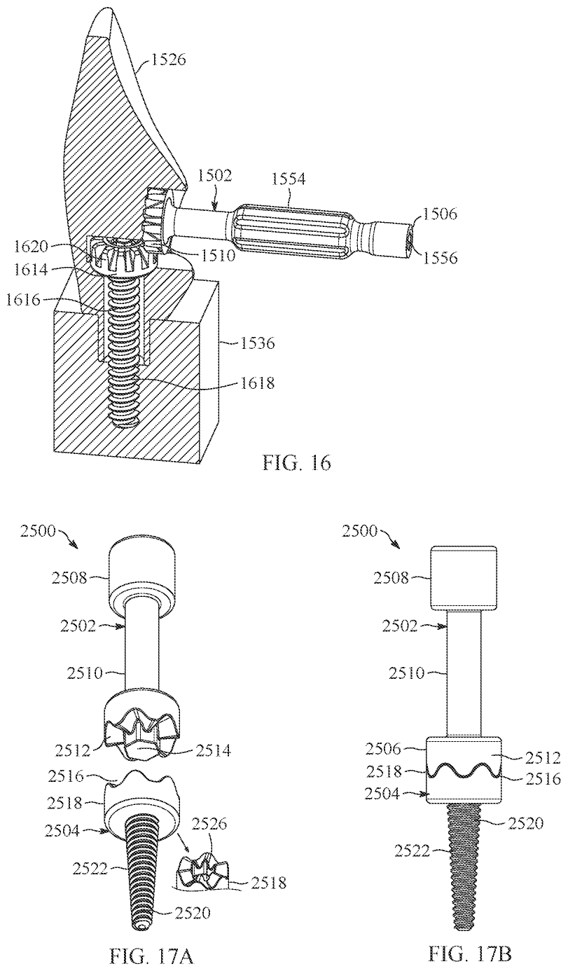

Referring now to FIG. 15 of the present invention illustrating a perspective view of a driving tool, in accordance with an alternate embodiment of the present invention. As shown in the figure, the driving tool 1502 is preferably a hand-driven tool and configured to be driven by a user manually. In this embodiment, the driving tool 1502 comprises a tip end 1504 and a tool head 1506 connected through a cylindrical shaft 1508. The cylindrical shaft 1508 also includes a gripping portion 1554 that facilitates the user to easily hold the driving tool 1502. The tip end 1504 includes a plurality of indentations 1510 configured to engage with the corresponding protrusions 1620 of the fastener 1612 shown in FIG. 16. In a preferred embodiment, especially in case of dental crown 1526 and abutment 1626 or when the first object 1534 has a recessed portion, the tip end 1504 of the driving tool 1502 provides stability during the fastening operation. The tip end 1504 stops the swivel, vertical, or lateral movement of the driving tool 1502 when engaged with a fastener 1612 at a particular angle. The circumferential part of the tip end 1504 contacts with the inner boundary of the recessed portion and thereby prohibits the swivel movement of the driving tool 1502 with respect to the fastener 1612. The tool head 1506 further includes a slot 1556 configured to receive any mechanical component such as shank, hex knob, etc., to drive the fastener and eject covering member.

Referring now to FIG. 16 of the present invention illustrating the engagement of driving tool 1502 (disclosed in FIG. 15) with a fastener 1612, in accordance with an exemplary embodiment of the present invention. In this embodiment, a dental crown 1526 is placed in the user's mouth via the fastening system of the present invention. There is shown a shank 1616 having a hexagonal-shaped end 1632 configured to be driven by the slot 1556 of the driving tool 1502. However, the one end of shank 1616 is not limited to hexagonal structure, it may include other shapes such as square, triangular, etc., depending upon the corresponding slot 1556 of the driving tool 1502. The driving tool 1502 drives the shank 1616 within the second object 1536, in that case, the second object 1536 is a gingiva consisting of the mucosal tissue.

Upon the insertion of the shank 1616 in the second object 1536, the dental crown 1526 is placed over the hexagonal-shaped end 1632 of the shank 1616. The dental crown 1526 includes a recessed portion for receiving the head 1614 of the fastener 1612. The head 1614 is inserted in the dental crown 1526 in such a way that head 1614 is centrally aligned with the hexagonal-shaped end 1636 of the shank 1616. In this embodiment, the head 1614 includes a through-and-through hole 1634 configured to receive the hexagonal-shaped end 1632 of the shank 1616. When the hexagonal-shaped end 1636 is partially inserted in the through-and-through hole 1634, the driving tool 1502 further rotates or drives the head 1614 in order to completely engage the shank 1616 with the head 1614 of the fastener 1612. Upon engagement, the dental crown 1526 is permanently fixed with the second object 1536. The tip end 1504 of the driving tool 1502 contacts the dental crown 1526 circumferentially during engagement and stops the swivel, vertical, or lateral movement of the driving tool 1502 when engaged and in function with a fastener 1612 at a particular angle.

In an embodiment of the present invention, a fastening system for holding two or more objects together, said fastening system comprises: a driving tool includes a driving tip and a tool head which are connected to each other through a cylindrical shaft, wherein the driving tip includes a plurality of teeth at its circumference and a protrusion member extended centrally from the driving tip; a fastener comprising a head whose lower surface is further connected to a shank, wherein the head of the fastener includes a plurality of drives and a receiving slot which are configured to receive the plurality of teeth and the protrusion member of the driving tool respectively; and a fastening cap having a plurality of teeth and is configured to cover or hides the upper portion of the fastener, when the fastener is securely placed by the driving tool between the objects that are meant to be held together; wherein the fastening cap has similar shape and dimension as the driving tip of the driving tool.

The fastener, such as a screw, nut, rivet, nail, sits flush with the first object with or without the cover that fastens two or more objects. The driving tool preferably engages with the fastener at a zero-degree angle, and the plurality of teeth of the driving tool is intimately and firmly engaged with the corresponding plurality of drives of the fastener.

In one embodiment of the present invention, the plurality of teeth of the driving tool includes but is not limited to V-shape, wave shape structure, helical structure, cycloidal structure, bevel structure, S-shaped structure, circular, square, rectangular, triangular, hexagonal, polygonal shape and similar; or any tapered configurations thereof. The protrusion member preferably has a hexagonal shape. However, the protrusion member is not particularly limited to hexagonal shape, it may include another shape such as V-shape structure, helical structure, cycloidal structure, bevel structure, S-shaped structure, wave-shaped, circular, square, rectangular, triangular, polygonal shape and similar; or any tapered configurations thereof.

In another embodiment of the present invention, the protrusion member either can be provided at the driving tip of the driving tool or can be provided at the head of the fastener.

In another embodiment of the present invention, the receiving slot either can be provided at the driving tip of the driving tool or can be provided at the head of the fastener. The fastening cap may also include a protrusion member that fits in the corresponding receiving slot of the fastener.

In one embodiment of the present invention, the fastening cap can be attached to the head of the fastener through a connecting mechanism. The connecting mechanism includes but is not limited to snap and fit mechanism, magnetic mechanism, glue, threaded mechanism, and the like. In some embodiments, the fastening cap is luted with the fastener's head through a sealing material. In some embodiments, the fastening cap serves as a secondary function such as hook, ring, slot, or any other type of connection.

The shank may include a plurality of male threads on its one portion, allowing the fastener insertion in the fastening channel when driven by the driving tool. The shank includes a smooth shaft portion and a sharp tip, allowing the nail fastener insertion between one or more objects when driven by a manual or motorized driving such as a hammer or a tapping device.

The plurality of drives of the fastener includes but is not limited to V-shape structure, helical structure, cycloidal structure, bevel structure, S-shaped structure, a wave-shaped, circular, square, rectangular, triangular, hexagonal, polygonal shape, and similar; or any tapered configurations thereof.

In an embodiment of the present invention, the head of the fastener is detachably attached with the shank through a connecting mechanism. The connecting mechanism between the head of the fastener and the shank preferably includes a threaded mechanism, however, it may include other connecting mechanisms such as snap and fit mechanism, magnetic mechanism, or a combination of fit mechanisms.

The driving tool is configured to be rotated either manually or automatically. A user can employ a hand-driven tool or a motorized tool such as a latch for the rotation of the fastener. In one embodiment, the driving tool is configured to be tapped or hammered against the fastener either manually or automatically. In that case, the user can employ a hand-driven tool such as a mallet against the driving tool or a motorized tool such as a nail gun.

The fastener is selected from the group containing non-permanent fasteners, wherein non-permanent fasteners include but are not limited to screw, nail, rivet, bolt fasteners, or any other threaded or non-threaded fasteners.

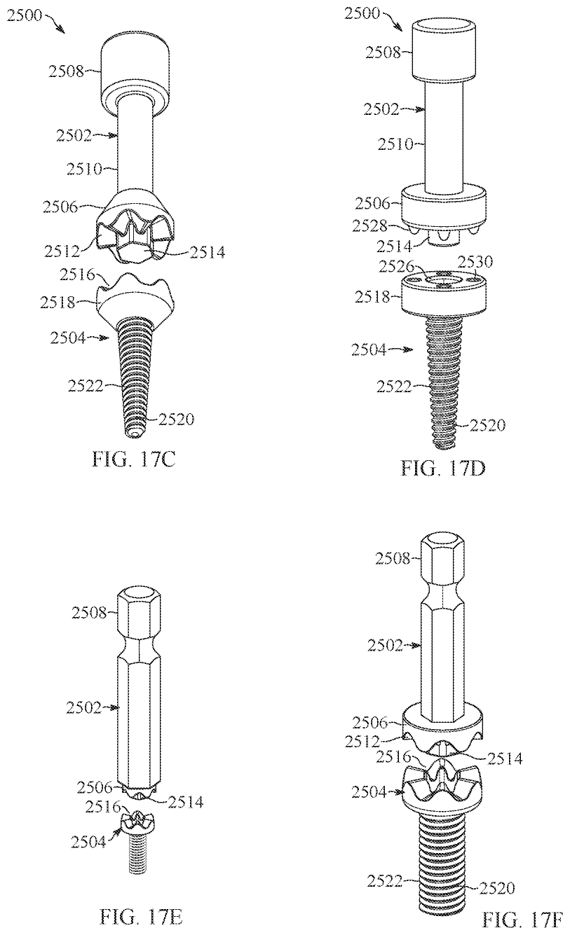

In another embodiment, the present invention discloses a fastening system for holding two or more objects together permanently or non-permanently. Referring now to FIG. 17a of the present invention illustrating a perspective view of a fastening system 2500 in accordance with an embodiment of the invention. There is shown a fastening system 2500 comprises a driving tool 2502 and a fastener 2504. The driving tool 2502 can be made of any solid material that offers high strength and rigidity during mechanical operation. In some embodiments, the driving tool 2502 is made of a rigid material like steel, iron, or any other material with nickel or chromium coating. In other embodiments, the driving tool 2502 can be made of stainless steel and titanium to ensure that bending or twisting deformation does not occur when applying force.

The driving tool 2502 of the present fastening system 2500 has a longitudinal body comprising two ends, wherein one end has a driving tip 2506, and the other end is a tool head 2508. Both ends of the driving tool 2502 are connected through a cylindrical shaft 2510 made of a solid and rigid material providing a high strengthening feature to the driving tool 2502. Depending on the application, the driving tool 2502 can be made of the same material as the fastener 2504 or of different materials. The driving tip 2506 includes a plurality of teeth 2512 positioned at its circumference. The plurality of teeth 2512 has a tapered U-shaped with a broader base than the tip with a slight curve at its ends that meshes with the corresponding plurality of drives 2516 of the fastener 2504 and generates a sufficient torque which is required for rotation. However, the plurality of teeth 2512 of the driving tool 2502 is not limited to tapered U-shaped structures, it may include other shapes as well, for example, helical, cycloidal, bevel, S-shaped, wave-shaped, circular, square, rectangular, triangular, hexagonal, polygonal shape and similar; or any tapered configurations thereof.

In some embodiments and depending on the application, the plurality of teeth 2512 and the plurality of drives 2516 can be made of a material like a cast iron, titanium, metal, solid material, aluminum alloy, brass, bronze that offers good mechanical properties during the operation of the fastening system 2500. Moreover, the plurality of teeth 2512 and the plurality of drives 2516 can also be made of a rigid plastic material such as ABS, polycarbonate, PPSU, rubber, and silicone, offering outstanding toughness during mechanical operations. Furthermore, in some embodiments, the plurality of teeth 2512 and plurality of drives 2516 can be made of a magnetic material, by which more enhanced griping or engagement is achieved between the driving tip 2506 and the fastener's head 2518. Moreover, in some embodiments depending upon the application area, the fastener 2504 such as screw, nails, rivets, bolts, etc., and the driving tool 2502 can be made of magnetic material or any other material by which intimate connection between the driving tool 2502 and the fastener 2504 is established. In these embodiments, the driving tool 2502 can act as a carrier to the fastener 2504 and tightly holds it in the air by the force of attraction and friction, reducing the chances of falling of the fastener 2504 from the driving tool 2502.

The driving tool 2502 further includes a protrusion member 2514 extended centrally from the driving tip 2506. The protrusion member 2514 is configured to engage with the corresponding receiving of the fastener 2504 and preferably has a hexagonal structure. However, the structure of the protrusion member 2514 is not limited to hexagonal shape, in other embodiments, it may include different shapes, for example, V-shape, helical, cycloidal, bevel, S-shaped, wave-shaped, circular, rectangular, square, triangular, and polygonal shape and similar; or any tapered configurations thereof.

Furthermore, in some embodiments, the fastening system 2500 includes a fastener 2504 composed of solid material such as low-medium carbon steel wire, brass, stainless steel, nickel alloy or aluminum, etc., which offers high strengthening durability during mechanical operation. In some embodiments, the fastener 2504 can be also be made of a material that is elastic, medical-grade, flexible with and/or without elasticity, rotatable, cost-efficient, easily manufacturable, plastically deformable, hygienic, sterile, tough, and/or lightweight. The fastener 2504 includes a head 2518 whose lower surface is connected to a shank 2520 having a plurality of threads 2522 including male or female threads on its one portion. The fastener 2504 further includes a plurality of drives 2516 having a U-shaped tapered U-shaped with a broader base than the tip with a slight curve at its ends and are formed on the head 2518. However, the plurality of drives 2516 of the fastener 2504 is not limited to U-shaped structure, it may include other shapes as well, for example, helical, cycloidal, bevel, S-shaped, wave-shaped, circular, square, rectangular, triangular, hexagonal, polygonal, and the like. The fastener 2504 also includes a receiving slot 2526 present on the center of its head 2518 and is configured to receive the protrusion member 2514 of the driving tool 2502. The receiving slot 2526 has a similar shape and dimension as protrusion member 2514 of the driving tool. During the engagement, the receiving slot 2526 tightly holds the protrusion member 2514. This arrangement between the receiving slot 2526 and the protrusion member 2514 enhances the engagement of driving tool 2502 with the fastener 2504 and produces sufficient friction and torque between them that is required for rotation. The functions of the described protrusion member 2514 and the receiving slot 2526 in the alternative connection configuration can be reversed for the fastener 2504 and driving tool 2502, depending on the application.

FIG. 17b illustrates the engagement of the driving tool 2502 and the fastener 2504 in accordance with an embodiment of the present invention. During engagement, the plurality of drives 2516 of the fastener 2504 engage or mesh with the plurality of teeth 2512 of the driving tool 2502 in such a manner that more friction is obtained between them and sufficient force for the rotation of the fastener 2504 is generated. The driving tool 2502 of the present invention is configured to be rotated or controlled manually or automatically. In some embodiments, the driving tool 2502 can be rotated by a hand-driven tool or a motorized latch. Depending upon the application area where the present fastening system is employed, for example, an area with tight spots having less working space, the tool head 2508 of the driving tool 2502 can be further connected to the driving shaft, which causes rotation of the driving tool 2502. Moreover, the clockwise rotation of the driving tool 2502 causes the fastener 2504 to be inserted within the hollow casing of the fastening channel 2524, and the anti-clockwise rotation of the driving tool results in the ejection or taking off the fastener 2504 from the fastening channel 2524.

In some embodiments, the driving tip 2506 and the head 2518 of the fastener 2504 may have a tapered structure as illustrated in FIG. 17c, wherein the plurality of teeth 2512 of the driving tool 2502 meshes or engages with the plurality of drives 2516 of the fastener 2504 accordingly. Furthermore, in alternate embodiments, as illustrated in FIG. 17d, the driving tip 2506 and the head 2518 of the fastener 2504 also have a flat structure. In this embodiment, the driving tip 2506 comprises a plurality of protrusion 2528 that meshes with the plurality of indentations 2530 of the fastener 2504, due to which sufficient force of friction is produced between the driving tool 2502 and the fastener 2504. Moreover, the plurality of protrusion 2528 and indentations 2530 can be provided on the circumferential part (outskirts area) of the driving tool 2502 and the fastener 2504, respectively.

In some embodiments, the plurality of indentions 2530 can stretch beyond or can be through-and-through holes of the fastener's head 2518. Further, the fastener 2504 also includes a receiving slot 2526 positioned at the center of the fastener's head 2518 and is configured to receive the corresponding protrusion member 2514 of the driving tool 2502. The plurality of protrusion 2528 and plurality of indentation 2530 can be of any size and taper (including low depth and high depth protrusion). In this embodiment, the plurality of protrusion 2528 and plurality of indentation 2530 preferably has a semi-spherical structure, however, they are not limited to semi-spherical shape; they may include other shapes such as hexagonal, circular, rectangular, square, polygonal, triangular as well. In one embodiment, the driving tool 2502 and the fastener 2504 may include micro and macro versions suitable for applications having compact and spacious working areas. FIG. 17e and FIG. 17f represent the micro-versions and macro-versions of the driving tool 2502 and fastener 2504, respectively, according to the alternate embodiment of the present invention.

Referring now to FIG. 18a of the present invention illustrating a perspective view of the fastening system 2600 in accordance with another embodiment of the invention. There is shown a driving tool 2602 comprising a driving tip 2604 and a tool head 2606 on its extreme ends, wherein both the driving tip 2604 and the tool head 2606 are connected through a cylindrical shaft 2608. The driving tool 2602 further includes a plurality of teeth 2610 provided at the circumference of the driving tip 2604 that engages with the corresponding plurality of drives 2614 of the fastener 2612. In this embodiment, the driving tool 2602 also includes a receiving slot 2616 (not shown) provided at the center of the driving tip 2604. The receiving slot 2616 is configured to receive the protrusion member 2618 of the fastener 2612. The protrusion member 2618 preferably has a hexagonal structure. However, the structure of the protrusion member 2618 is not limited to hexagonal shape, in other embodiments, it may include other shapes, for example, circular, rectangular, square, triangular, and polygonal similar; or any tapered configurations thereof.

Referring now to FIG. 18b of the present invention illustrating the engagement between the driving tool 2602 and the fastener 2612 in accordance with an embodiment of the invention. During engagement, the plurality of teeth 2610 of the driving tool 2602 completely meshes or engages with the plurality of drives 2614 of the fastener. Also, the protrusion member 2618 of the fastener 2612 is received by the receiving slot of the driving tool 2602. Therefore, when the driving tool 2602 is rotated by the external force that can be applied manually or automatically, a force of friction is produced between the plurality of teeth 2610 and the plurality of drives 2614. The produced force rotates the fastener 2612, and their rotational motion causes the insertion of fastener 2612 within the fastening channel 2524 or the removal of the fastener 2612 from the fastening channel 2524.

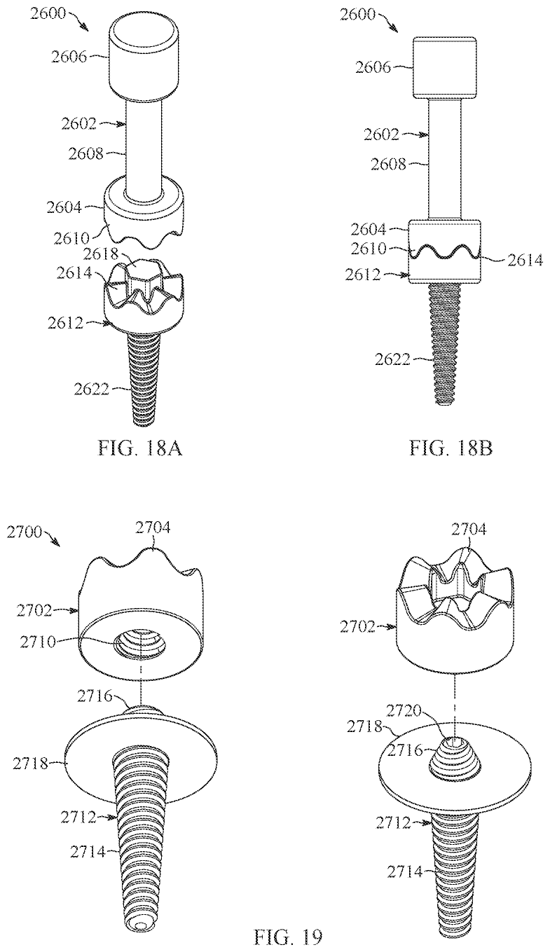

Referring now to FIG. 19 of the present invention illustrating an exploded view of the fastener in accordance with an embodiment of the invention. There is shown a fastener 2700 comprising a head 2702 having a plurality of drives 2704 on the top surface of the head. The plurality of drives 2704 is configured to engage with the plurality to teeth 2512 of the driving tool 2502. The head 2702 of the fastener 2700 further includes a plurality of inner threads 2710 positioned at the inner surface of the fastener 2700. The plurality of inner threads 2710 can be of any shape and size, including circular, helical, blade, constant pitch, V-threads, etc. The recessed portion of the head 2702 can be of any depth or configuration, through-and-through, or could be a projection of any shape to serve the same purposes via engagement with the shank 2712.

Furthermore, there is shown a shank 2712 and is configured to detachably attached with the head 2702 of the fastener 2700 through a threaded mechanism. However, in some embodiments, the head 2702 of the fastener 2700 can be detached with the shank 2712 using other connecting mechanisms such as magnetic, glue, snap, and fit mechanisms. The shank 2712 further includes a plurality of outer threads 2714 on its outer surface and preferably has a conical structure on its one extreme end 2716 that goes inside the fastener's head 2702 when attached. The extreme end 2716 of the shank 2712 further includes a recess 2720 that receives a driving key such as a hex key whose function is to remove the shank 2712 from the fastening channel or area. The recess 2720 preferably has a hexagonal structure that receives the one end of the driving key and provides access for further rotation. However, the recess 2720 is not limited to hexagonal structure, it may include other structures also such as round, square, rectangle, trilobed, polygonal, etc. The recess 2720 of the shank 2712 can be of any depth or could be a projection of any shape to serve the same purpose via a wrench-like removal mechanism. Further, the shank 2712 may include a washer 2718 that is provided at one end of the shank 2712. The washer 2718 can be made of a solid material such as hardened steel, metal, plastics, rubbers, etc., and is configured to prevent the loss of pre-load due to brinelling after the torque is applied. By using washer 2718 in the present fastening system, the bearing surface pressure is dispersed so that the risk of bearing surface sinking and the resulting looseness can be reduced. The washer 2718 also helps in preventing galvanic corrosion, particularly by insulating steel screws from aluminum surfaces.

Referring now to FIG. 20 of the present invention illustrating a sectional view of fastening system 2500 when a first object 2802 and a second object 2804 joined together permanently or non-permanently in accordance with an embodiment of the invention. The first object 2802 and the second object 2804 can be of any shape, size and these are the objects that need to be fastened together using the present fastening system 2500. There is shown a driving tool 2502 engages with a fastener 2504 at a zero-degree engagement angle in a straight line. At a zero-degree angle, the plurality of teeth 2512 of the driving tool 2502 completely meshes with the plurality of drives 2516 of the fastener 2504, and less slipping has occurred between the tools. Due to which more frictional and gripping force is achieved between them. Furthermore, there is shown a protrusion member 2514 provided at the driving tip 2506 of the driving tool 2502 that is entirely received by the receiving slot 2526 of the fastener 2504. This arrangement between the protrusion member 2514 and the receiving slot 2526 improves the friction between the driving tool 2502 and the fastener 2504 and also helps in reducing the stripping, slipping problems of the fasteners. Depending on the application, the plurality of drives 2516 of the fastener 2504 can be placed inside the first object 2802, can be flush with the first object 2802, or can be placed above the first object 2802. In some embodiments, the plurality of drives 2516 or the fastener's head 2518 may or may not be covered by a cap depending on the application area of the present fastening system 2500.

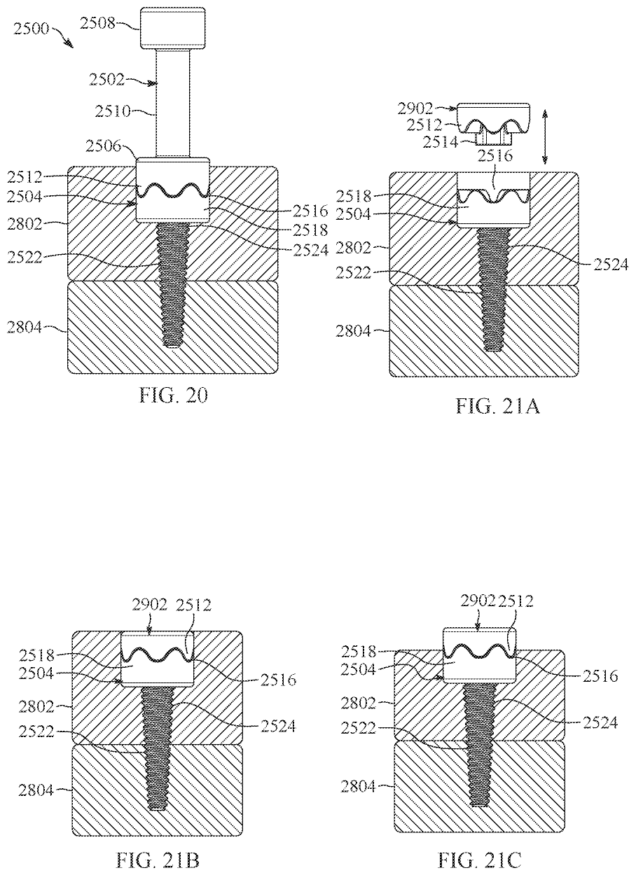

Referring now to FIG. 21A and FIG. 21B of the present invention illustrating a fastening cap 2902 engaged with the head 2518 of the fastener 2504, in accordance with another embodiment of the present invention. A fastening cap 2902 is configured to hide or cover the head 2518 of the fastener 2504. The fastening cap 2902 can be made of any solid material, for example, stainless steel, aluminum, nylons, soft metals, polypropylene, polytetrafluoroethylene (PTFE) material, or as the same material of the fastener 2504 or the driving tool 2502. The fastening cap 2902 provides good corrosive resistive properties and good chemical, mechanical properties with or without coatings, which are affected by outer environment conditions like rain, dew, moisture, etc. In some embodiments, the fastening cap 2902 can also be made of resin composites material whose function is to cover or hide the head 2518 of the fastener 2504 or can be formed with pre-fabricated configurations to restore the esthetic features of the system. The fastening cap 2902 further includes a plurality of teeth 2512 positioned on its circumference and a protrusion member 2514 centrally extended from the fastening cap 2902. As shown in FIG. 21A, the protrusion member 2514 has a hexagonal structure which is received by receiving slot 2526 of the fastener 2504. In alternate embodiments, the protrusion member 2514 may include other shapes, for example, circular, rectangular, square, triangular, and polygonal shape and similar; or any tapered configurations thereof. The functions of the plurality of drives 2516 and the plurality of teeth 2512 in the alternative configuration can be reversed depending on the application.

The fastening cap 2902 has a similar shape, size, and dimension as the driving tip 2506 of the driving tool 2502 has. More particularly, the size and shape of the fastening cap 2902 depend upon the corresponding plurality of drives 2516 and the receiving slot 2526 of the fastener 2504. For proper engagement, it is necessary that the plurality of teeth 2512 and protrusion member 2514 of the fastening cap 2902 completely meshes with the plurality of drives 2516 and the receiving slot 2526 of the fastener 2504, respectively. The fastening cap 2902 also helps in maintaining the stability of the fastener 2504 within the fastening channel 2524. FIG. 21B illustrates a fastener 2504 whose head 2518 is entirely covered by the fastening cap 2902 and is flushed within the objects which are fastened together. However, it is not always necessary that the fastener's head 2518 is completely flushed within the first object 2802. In some embodiments, as shown in FIG. 21C, the fastener's head 2518 and fastening cap 2902 may also be positioned above the top surface of the first object 2802 and is available for rotation in a clockwise direction as well as anti-clockwise rotation. In this embodiment, the fastening cap 2902 and the head 2518 of the fastener 2504 can be removed using a removing tool. In other embodiments, the fastening cap 2902 may be left out for the fastener 2504 and can be flushed with the top surface of the first object 2802 or can be placed above the first object 2802.

The fastening cap 2902 helps in maintaining the structural integrity of the fastener 2504 and restores the aesthetic features of the fastener 2504. Moreover, in some embodiments, the fastening cap 2902 is connected to the head 2518 through a connecting mechanism that allows the removal of the fastening cap 2902 from the head 2518. When the fastening cap 2902 is removed from the fastener's head 2518, the fastener 2504 can be unscrewed from the fastening channel 2524 and is available for reuse. The fastening cap 2902 can be connected to the head 2518 of the fastener 2504 using a snap and fit mechanism, magnetic mechanism, glue, threaded mechanism, and the like. In another embodiment, the fastening cap 2902 is luted with the fastener's head through a sealing material. The fastening cap 2902 can be made of breakable material such as plastic, glass, rubber that can be torn or break away from the fastener's head 2518 and makes the plurality of drives 2516 visible for screwing or unscrewing purposes. In one embodiment of the present invention, the fastening cap 2902 may include a slot or a hook connection on the upper surface of the fastening cap 2902 that facilitates the easy removal or ejection of the fastening cap 2902 from the head 2518 of the fastener 2504.

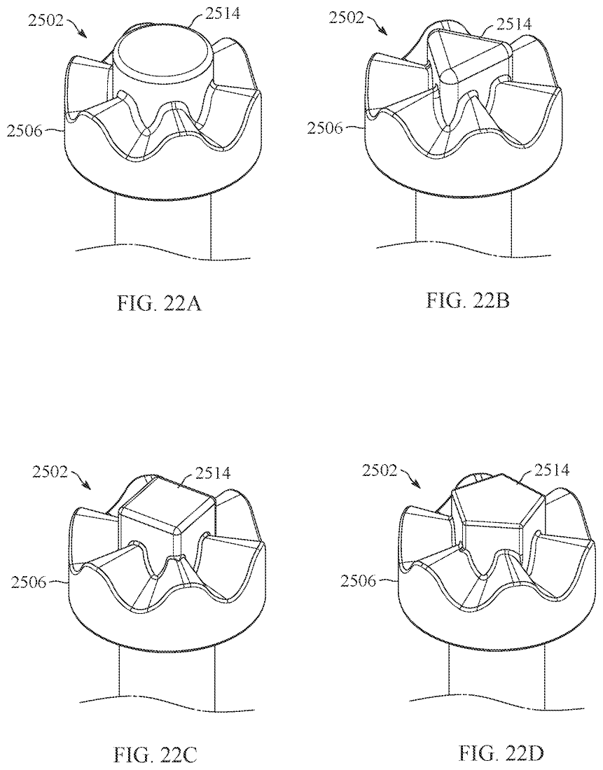

Referring now to FIG. 22A illustrating a perspective view of the driving tip 2506 of the driving tool 2502 in accordance with an exemplary embodiment of the present invention. In this embodiment, the protrusion member 2514 of the driving tip 2506 has a circular structure that fits or engages with the receiving slot 2526 of the fastener 2504. Other shapes of protrusion member 2514, such as triangular, circular, and polygonal, are illustrated in FIG. 22B, FIG. 22C, and FIG. 22D. Similarly, the fastener 2504 can include a receiving slot 2526 identical to the protrusion member 2514 of the driving tip 2506. It is important to note that all configurations including protrusion member 2514, receiving slot 2526 in the complex, can be interchangeable and may or may not have a taper for better engagement and removal.

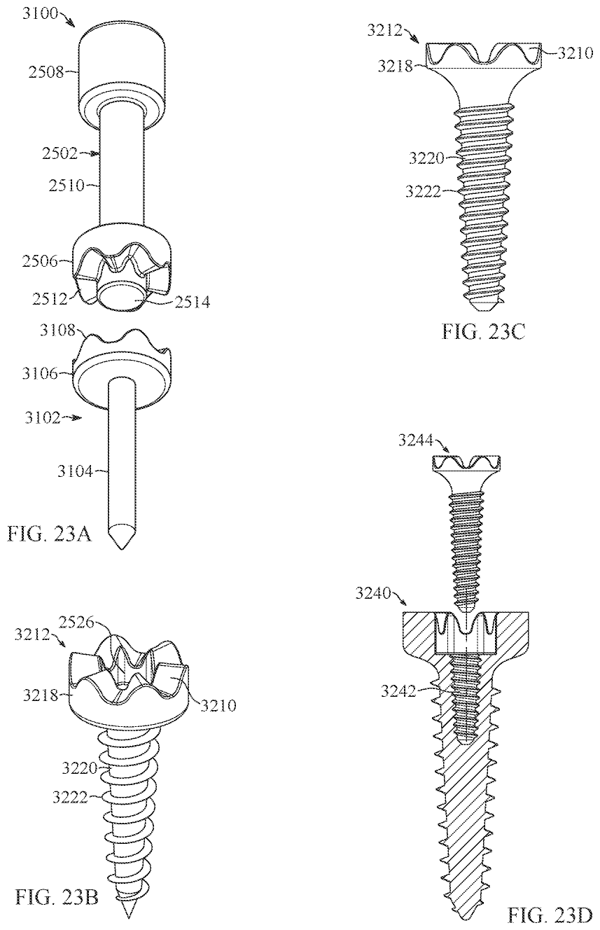

Referring now to FIG. 23a of the present invention illustrating a perspective view of the fastening system 3100 in accordance with an embodiment of the invention. In this embodiment, a non-threaded fastener such as nail fastener 3102 is being employed as a fastener for fastening purposes. However, the present invention is not limited to nail fastener 3102, it may include other non-threaded fasteners such as square keys, tapper pins, cotter pins, dowel pins, etc. In one embodiment, the fastener 3212 can be a deep thread fastener and a thin head fastener, as illustrated in FIG. 23b and FIG. 23c, respectively. The nail fastener 3102 includes a shank 3104, a long stem part of the nail fastener 3102, and a head 3106 provided on the top of the shank 3104. The shank 3104 of the nail fastener 3102 has a tip or sharp end at the bottom. The head 3106 further includes a plurality of drives 3108 and is configured to engage or mesh with the plurality of teeth 2512 of the driving tool 2502. In some embodiments, the head 3106 of the nail fastener 3102 also includes a receiving slot 2526 can be of any size and shape (circular, rectangular, square, hexagonal, triangular, and polygonal, or have a taper) that receives the corresponding protrusion member 2514 of the driving tool 2502.

The plurality of teeth 2512 of the driving tool 2502 and the plurality of drives 3108 of the nail fastener 3102 are perfectly engaged in a way that driving tool 2502 can easily carry and hold the nail fastener 3102 in the air by the force of friction and act as a carrier of the nail fastener 3102. The nail fastener 3102 can also be held by the driving tool 2502 by other connecting mechanisms, for example, magnetic connection, glue, and snap-fit connection. The nail fastener 3102 can be fastened to the first object 2802 and second object 2804, using a hammer, mallet, or any pounding force produced by a motor. The hammer, mallet, or pounding force can be applied to the driving tool 2502, and this force is transferred to the nail's head 3106, thereby causing the insertion of the nail fastener 3102 within the objects. This embodiment is useful in areas where shear strength is needed, and the material has sideways pressure or would have the tendency to move left or right. Then a nail fastener 3102 is required for the fastening process.

Referring now to FIG. 23B of the present invention illustrating a deep thread fastener 3212 according to an alternate embodiment of the present invention. In this embodiment, the fastener 3212 includes a shank 3220 having a reduced diameter along the length and is tapered in a conical form at one end. The shank 3220 further includes a plurality of male thread 3222 offers more resistance to pull-out forces as compared to standard wood and steel screws. The deep thread fastener 3212 enables self-tapping and more effective insertion of fastener 3212 with or without a pre-drilled site. Referring now to FIG. 23C of the present invention illustrating a front view of a fastener having a thin head for engagement in accordance with an alternate embodiment of the present invention. In alternate embodiments, the fastener 3212, as mentioned in FIGS. 23B and 23C, may include a cavity positioned on the head 3218 of the fastener 3212. The cavity facilitates the engagement of the fastener 3212 with other bodies via magnetic, friction retained, or any other type of connection to perform a specified task.

Referring now to FIG. 23D of the present invention illustrating a fastener 3240 (0-degree fastener) having an internal thread channel 3242 for receiving a secondary threaded fastener 3244, in accordance with an alternate embodiment of the present invention. In this embodiment, the internal thread channel 3242 is utilized for secondary purposes, such as for placing an object over the fastener 3240. In one embodiment, the fastener 3240 facilitates the positioning of the dental crown or another object on the head of the fastener 3240. In addition, the threaded connection between the fastener 3240 and the secondary threaded fastener 3244 helps achieve more force of friction and improves the stability between one or more mechanical components.

In an embodiment of the present invention, a fastening system comprising a universal jammer having a plurality of indentations that meshes or engages with a plurality of protrusion of the fastener at multiple angles is provided. Basically, the universal jammer is configured the same as the tip end of the matching driving tool. The tip end of the driving tool acts as a jammer when detached from the cylindrical shaft of the driving tool. Universal jammer configured to be employed in such scenarios where the head of the fastener is not accessible, for example, areas with tight spots and where the head of the fastener is flushed within the objects. The universal jammer includes a groove provided at the bottom portion of the jammer and is configured to receive a driving shaft, for example, a hex key that rotates the jammer in a clockwise and anti-clockwise rotation. During the engagement position, the indentation of the jammer firmly meshes with the protrusion of the fastener. The rotational motion of the jammer causes the insertion and removal of the fastener accordingly. It is important to note that this complex can be housed via a solid material to hold all components firm in their relationship to form a hand tool that can be rotated in a horizontal plane while the fastener head and bits and adaptors can act as the vertical driving force for any fastener connection such as bolts and other type of nails and screws.

In another embodiment of the present invention, a universal nut is provided, which is being used with the conjugation of a bolt fastener in order to join objects together. The universal nut comprises a circular body with a top surface and a bottom surface. The top surface includes a plurality of protrusions that mesh or engage with a plurality of indentations of the driving tool. The universal nut further comprises a threaded hole that allows the rotation of the universal nut over the bolt fastener. More specifically, the universal nut includes a plurality of internal threads that engage with the plurality of external threads of the bolt fastener. During working, when the plurality of indentations of the driving tool meshes with the plurality of protrusions of the universal nut, a sufficient force of friction and torque is generated between them, causing the rotation of the universal nut. Thus, the clockwise rotation of the universal nut causes the tightening of the fastener, and the anti-clockwise rotation of the universal nut causes the loosening of the fastener. Thus, in one embodiment, the driver permits the bolt fastener to enter as the nut is driven, creating a guide system for driving the nut more efficiently and effectively.

In yet another embodiment of the present invention, a universal lifter is provided. The lifter is configured to cut the material circumferentially and drills the material centrally. The material can be of flexible or solid nature, for example, wood, bone, plastic, iron, concrete, aluminum, copper, and the like. The universal lifter can also be applied in the medical field for bone and other solid living tissue, such as dentistry for lifting sinus membrane in order to fasten a dental implant. In dentistry, the universal lifter cuts the bone circumferentially and pushes the drilled bone part towards the sinus membrane. The universal lifter can vertically or horizontally lift the solid material to the desired height or length or beyond the core by adding like materials before, during, or after the drilling before placement or securing of the fastener.

The universal lifter includes a cylindrical body having a plurality of cutting blades positioned on one end of the cylindrical body. The blade ends can be configured in any shape: saw-like, modified saw patterns, rounded, wave-like, pointy, triangular, rectangular, polygonal, or similar. The plurality of cutting blades have sharp edges configured to cut the material circumferentially. The plurality of cutting blades can be made of solid materials such as stainless steel, cobalt, diamond, carbide material providing high strength during the cutting operation. In some embodiments, especially in the medical field, the plurality of cutting blades are made of a non-reactive, inert, biocompatible material such as titanium. The cylindrical body includes a bore extending therethrough for receiving the material, which is circumferentially cut by the plurality of cutting blades.

The universal lifter may further include one or more drill bits centrally positioned on the distal end of the cylindrical body. The drill bit performs the drilling operation over the material that is circumferentially cut by the plurality of cutting blades. The drill bit includes a plurality of blades that cuts material thoroughly and makes a sufficient space in the material to receive a fastener. The purpose is to create an engaging or a non-engaging region to receive the lifting fastener. The drill bit is preferably made from the same solid materials as the rest of the instrument. In some embodiments, the drill bit can also be made of low- and high-grade carbon steel, carbide, cobalt, PCD (polycrystalline diamond), or similar solid metals. The cylindrical body and the drill bit are configured to be rotated by a driving shaft driven by the hand-held driver with a latch adaptor or motorized tool that can receive a latch-type driver or any other driving mechanism. The driving shaft is extended from the distal end of the cylindrical body. The universal lifter may have embodiments with lines, demarcations, colorations, and/or indentations that inform the operator of the length the lifting tool has been advanced. They may be metric or imperial measurement lines alongside the partial or entire length of the instrument.

During working, the plurality of cutting blades cut the material circumferentially until the desired length. The drill bit performs the drilling operation over the material and prepares the site to receive a fastener. During drilling operation, the drilled or excess material is pushed towards the wall of the cylindrical body by the drilling unit and its vents, and side by side it densifies the adjacent material with the same material. Thereafter, a fastener such as a crew can be fastened to the denser material. The material is lifted or pushed to the desired height when the driving tool rotates the fastener against the denser materials and circumferentially larger weakened base by the cutting tip. In dentistry, for example, the sinus membrane is lifted by the bone core left behind, which is pushed by the rotational force of the fastener, in a controlled fashion to lift the bone core/membrane complex. In an alternative embodiment, the same-like material can be added when the material thickness is too short and does not reach the drill bit of the universal lifter. When the desired drilling length is reached, the core is separated from the body by the cutting edge of the universal lifter.

In another embodiment of the present invention, a handheld universal driver is provided for tightening and loosening of different types of fasteners such as screw, bolt, flathead, star head, hex head fasteners, and the like. The handheld universal driver can be applied in those scenarios or areas where there is no space to drive the fastener. By using the handheld universal driver, fastening of objects would be easier in inaccessible areas. The handheld universal driver can be made of any solid material such as cast iron, titanium, metal, solid material, aluminum, alloy, brass, bronze, and any other material having high strengthening features. The handheld universal driver is configured to be driven by a battery or a power source provided in the housing of said driver. In one embodiment, the handheld universal driver can also be driven by a user manually via the handle.

The handheld universal driver includes a handle that is rotatably connected to a housing. The handle is free to rotate clockwise and counterclockwise while the housing remains fixed. In some embodiments, the housing is connected to the handle part through a bearing component that allows the 360-degree rotation of the handle while the housing remains fixed. In alternate embodiments, the handheld universal driver may also employ a ratchet mechanism or any other mechanism that can amplify the torque applied to the driving rod. The handle may include a patterned surface that allows the user to grip or hold the handheld universal driver more efficiently.

The handle is further connected to a driving rod configured to drive an adaptor provided in the housing of a handheld universal driver. In a preferred embodiment, the driving rod is permanently attached or fixed to the handle and rotates when the handle is turned. The handle portion and the driving rod can rotate 360-degree in clockwise and anti-clockwise directions to drive the adaptor. The driving rod further includes a plurality of indentations provided at one end of the driving rod, engaging with the plurality of protrusion of the adaptor. During engagement, when the driving rod is rotated in the horizontal plane with the help of the handle, the adaptor is forced to rotate in a vertical plane, causing the rotation of bit of any shape attached to the adaptor. The adaptor has a similar configuration as the head of the fastener mentioned in the above aspects of the invention. The adaptor may also include an anti-skidding element that maintains steady rotation of the driving rod over the plurality of protrusion of the adaptor.

In some embodiments, the handheld universal driver may employ a detachable connection that allows the replacement of the adaptor when needed. In this embodiment, the handheld universal driver includes a cover that is detachably attached to the housing through a connecting mechanism. The cover is provided above the adaptor and can be removed once the replacement is needed. For replacement, the handle, which is permanently attached to the driving rod, moves backward, causing the disengagement of the driving rod and the adaptor. Then, the cover is detached from the housing, and the adaptor is removed. In alternate embodiments, the cover can also act as the anti-skidding element to maintain the efficient rotation of the adaptor.

The adaptor further includes an opening configured to receive a bit rotated by the handheld universal driver. The bit firmly engages with the adaptor and turns in the same direction in which the adaptor is rotated. The bit can be attached to the adaptor through a connecting mechanism. The connecting mechanism includes but is not limited to snap and fit mechanism, magnetic mechanism, friction connection, and quick release mechanism. The clockwise and anti-clockwise rotation of the bit by the handheld universal driver causes the tightening and loosening of the fastener, respectively. The top of the fastener engages with the bottom end of the bit. In one embodiment, the bit has a hexagonal structure that matches with the configuration of the opening of the adaptor, however in alternate embodiments, it may include a bit of different shapes such as circular, square, rectangular, triangular, hexagonal, polygonal shape, and similar; or any tapered configurations thereof.

In some scenarios, the handheld universal driver may also include a universal jammer (mentioned in the above aspect of the invention) comprising a plurality of indentations that engages with the plurality of protrusions of the adaptor. In these embodiments, the driving rod may include a hexagonal structure at one end instead of the plurality of indentations. During operation, the hexagonal end of the driving rod engages with the respective receiving of the jammer. It causes the rotation of the universal jammer in the same direction in which the driving rod is rotated. The rotational motion of the universal jammer by the driving rod in the horizontal plane further causes the rotation of the adaptor in the vertical plane.

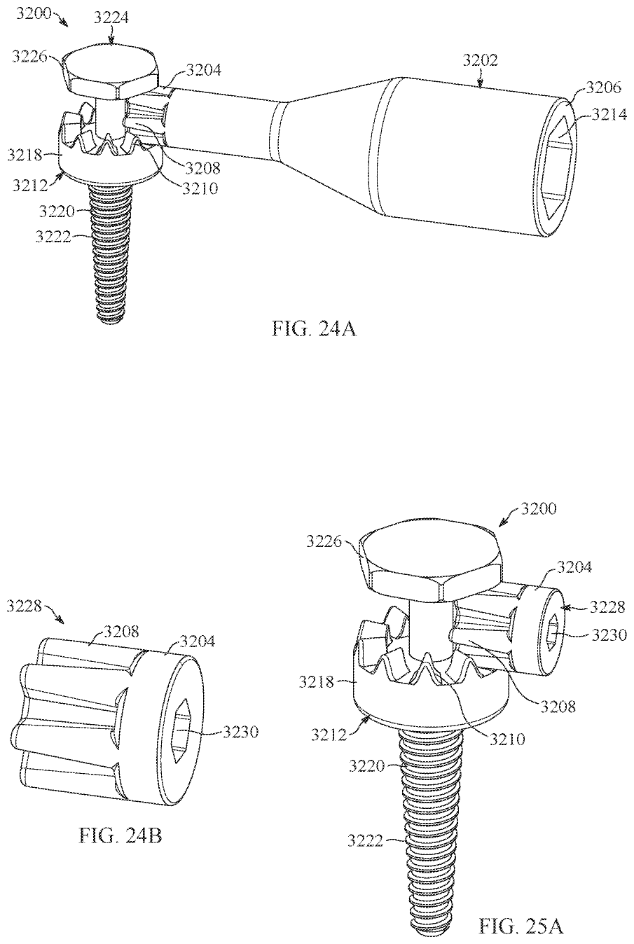

In another embodiment, the present invention discloses a fastening system for joining, anchoring, or holding two or more objects together, where a driving tool engages with a fastener at 0 degrees and/or various other angles. Referring now to FIG. 24a of the present invention illustrates a perspective view of the fastening system 3200 according to an embodiment. There is shown a driving tool 3202 comprising a longitudinal body having a first end and a second end, wherein the first end is a tip end 3204 and the second end is a tool head 3206. The driving tool 3202 is preferably made of solid material that offers high strength and rigidity during mechanical operation. In some embodiments, the driving tool 3202 is made of a rigid material like steel, iron, or any other material with nickel or chromium coating. In other embodiments, the driving tool 3202 can be made of stainless steel and titanium to ensure that bending or twisting deformation does not occur when applying force. In other applications, 3202 can be made out of any solid materials such as metals, plastics, ceramics, rubbers, silicones, and similar. The tip end 3204 of the driving tool 3202 includes a plurality of indentations 3208 that meshes or engages with a plurality of protrusion 3210 of a fastener 3212. The plurality of indentations 3208 of the driving tool 3202 can be made of any solid material such as cast iron, titanium, metal, solid material, aluminum, alloy, brass, bronze that offers good mechanical properties during the operation of the fastening system 3200. In some embodiments, based on the application area where the present fastening system 3200 is being employed, the plurality of indentations 3208 can be made of a rigid plastic material such as ABS, polycarbonate, PPSU, rubber, and silicone. These materials offer high resistivity and strength during mechanical operations.

The plurality of indentations 3208 of the driving tool 3202 preferably has a U-shaped structure with less width at the bottom side, gradually increasing towards the top, such as a Wave-shaped configuration. However, the plurality of indentations 3208 is not limited to a U-shaped structure. It may include other shapes, such as V-shape, helical, cycloidal, bevel, S-shaped, wave-shaped, circular, square, rectangular, triangular, hexagonal, polygonal shape, and similar; or any tapered configurations thereof. The tool head 3206 of the driving tool 3202 is configured to be attached with a motorized tool for driving purposes. In some embodiments, the driving tool 3202 can also be driven by a hand-driven tool. Further, a receiving slot 3214 is shown on the bottom portion of the driving tool 3202 that facilitates ejection of the anti-skidding element 3224 from the head 3218 of the fastener 3212, according to one embodiment of the invention.

The fastening system 3200 further includes a fastener 3212 composed of any solid material such as metals, ceramics, plastics, rubbers, silicones, low-medium carbon steel wire, brass, stainless steel, nickel alloy, aluminum, etc., that offers high strengthening and durability during mechanical operation. In some embodiments, the fastener 3212 can also be made of a material that is elastic, medical-grade, flexible with or without elasticity, cost-efficient, easily manufacturable, plastically deformable, hygienic, sterile, tough, and/or lightweight. The fastener 3212 includes a head 3218 containing a plurality of protrusion 3210 positioned on the upper surface of the head 3218. The plurality of protrusion 3210 is made of the same material as the plurality of indentations 3208. The plurality of protrusion 3210 may have different shapes such as U-shape, Wave-shape, V-shape, helical, cycloidal, bevel, S-shaped, wave-shaped, circular, square, rectangular, triangular, hexagonal, and polygonal shape.

The fastener 3212 further includes a shank 3220 centrally fixed to the head 3218, comprising a plurality of male threads 3222 on its one portion and enables the insertion of the fastener 3212 within a fastening channel or area where the fastener 3212 is rotated by the driving tool 3202. In alternate embodiments, the plurality of male threads 3222 may have a shape like deep threads, as shown in FIG. 23b, enabling self-tapping and more effective insertion of fastener with or without a pre-drilled site. The shank 3220 of the fastener 3212 can be tapered or straight in length, similar to a bolt. In some embodiments, the shank 3220 is detachably attached with the head 3218 of the fastener 3212 via a connection mechanism. The connection mechanism may include a snap and fit mechanism, magnetic mechanism, cement, and threaded mechanism for detachment. During engagement, the plurality of indentations 3208 of the driving tool 3202 effectively engages with the plurality of protrusion 3210 of the fastener 3212 and generates a sufficient force of friction between them to rotate the fastener 3212.

Furthermore, the fastener 3212 includes an anti-skidding element 3224 positioned on the head 3218 of the fastener 3212. The anti-skidding element 3224 is also made of any solid material, for example, ceramics, plastics, rubbers, silicones, and metals, offering good strengthening properties during the engagement between the driving tool 3202 and the fastener 3212. The anti-skidding element 3224 further maintains the position of the driving tool 3202 during fastening operations. The anti-skidding element 3224 prohibits the slipping of the driving tool 3202 over the head 3218 of the fastener 3212 and maintains steady rotation. In some embodiments, the anti-skidding element 3224 is detachably attached with the head 3218 of the fastener 3212. The anti-skidding element 3224 can be detached via the threaded mechanism, however, it may include other mechanisms such as magnetic mechanism, cement or glue, snap and fit mechanism for detachment purposes. The anti-skidding element 3224 consists of a top 3226 that may have a plurality of shapes such as square, hexagonal, circular, polygonal, etc., depending on the application.

Referring now to FIG. 24b of the present invention illustrating a perspective view of the universal jammer 3228 in accordance with one embodiment. The universal jammer 3228 is basically the tip end 3204 of the driving tool 3202, as illustrated in FIG. 24a of the present invention. The universal jammer 3228 can be made of any solid materials such as metals, ceramics, plastics, rubbers, and silicones. The universal jammer 3228 is configured to be employed in such scenarios where the head 3218 of the fastener 3212 is not accessible, or the fastening complex is jammed and stabilized. Inaccessible areas, for example, tight spots and where the head 3218 of the fastener 3212 is driven from the side. More specifically, in situations where the plurality of protrusion 3210 of fastener 3212 are not visible or cannot be accessed by the driving tool 3202 for driving purposes.

The universal jammer 3228 includes a plurality of indentations 3208 that meshes or engages with the plurality of protrusion 3210 of the fastener 3212. The universal jammer 3228 further consists of a groove 3230 provided at the bottom surface of the universal jammer 3228. The groove 3230 is configured to receive a driving shaft such as a hex key that rotates the universal jammer 3228 in a clockwise and anti-clockwise rotation. The rotational motion of the universal jammer 3228 causes the further insertion or removal of fastener 3212 accordingly. The groove 3230 of the universal jammer 3228 can also be a projection-type shape, or a flat surface, threaded or patterned surface for better engagement with the driving shaft and could be of any shape as a circular, square, rectangular, triangular, hexagonal, polygonal shape, and similar; or any tapered configurations thereof.

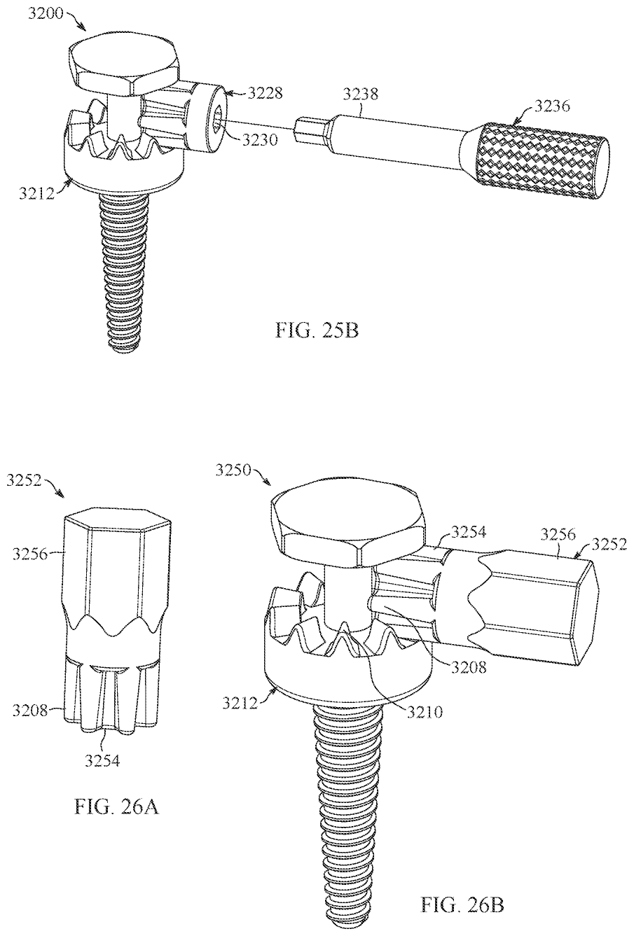

Referring now to FIG. 25a of the present invention illustrates the engagement of the universal jammer 3228 with the fastener 3212 according to one embodiment. As shown in the figure, the universal jammer 3228 engages with the fastener 3212 at a 90-degree angle. However, in the present invention, the angle between the universal jammer 3228 and the fastener 3212 is not limited to a 90-degree angle. In some embodiments, it may include 0-degree, 45-degree, and 135-degree engagement. The universal jammer 3228 can be rotated by any driving mechanism, for example, it is rotated by the driving shaft of a hand-driven tool or a motorized tool. During engagement, when the driving shaft engaged with the groove 130 and rotates the universal jammer 128 in a clockwise direction, thereafter the fastener 112 tends to rotate in the clockwise direction as well causing the insertion of the fastener 112 within the fastening channel or area where the fastening system 100 is employed.

Referring now to FIG. 25b of the present invention illustrating a perspective view of a fastening system when the universal jammer 3228 is driven by a handheld driver 3236 in accordance with an alternate embodiment of the invention. There is shown a universal jammer 3228, which is in an engaged position with the fastener 3212. On its bottom portion, the universal jammer 3228 includes a groove 3230 having a hexagonal shape, and it receives the driving shaft 3238 of the handheld driver 3236. The driving shaft 3238 also has a hexagonal structure and engages with groove 3230 firmly and intimately. The handheld driver 3236 is configured to rotate along the horizontal plane, causing the rotation of the fastener 3212 within the fastening channel. For tightening the fastener 3212, the handheld driver 3236 needs to be rotated in a clockwise direction, and for loosening purposes, it should be rotated in the anti-clockwise direction.

Referring now to FIG. 26a of the present invention illustrating a perspective view of a driving bit in accordance with an embodiment of the present invention. There is shown a driving bit 3252, which can be made of any solid material or the same material as the driving tool 3202 mentioned in the above embodiments. The driving bit 3252 comprises a front end 3254 and a distal end 3256. The front end 3254 includes a plurality of indentations 3208 that meshes or engages with the plurality of protrusion 3210 of fastener 3212 during the fastening process. The distal end 3256 of the driving bit 3252 is configured to be detachably attached with the hand-driven or motorized tool through a connecting mechanism. The connecting mechanism includes but is not limited to the snap and fit mechanism, magnetic mechanism, friction, or similar. The distal end 3256 of the driving bit 3252 preferably has a hexagonal-shaped structure, however, in some embodiments, it may include other shapes such as circular, triangular, square, rectangular, elliptical, rectangular, polygonal as well.

Referring now to FIG. 26b of the present invention illustrating a perspective view of the fastening system 3250 when the driving bit 3252 engages with the fastener 3212 in accordance with an embodiment of the invention. During engagement, the plurality of indentations 3208 of driving bit 3252 firmly meshes with the plurality of protrusion 3210 of fastener 3212 and thereby generates a sufficient force of friction between the elements and causing the rotation of the fastener 3212. The driving bit 3252 is configured to be rotated by a hand-driven tool or a motorized tool. In some embodiments, the driving bit 3252 can be rotated by a latch motor.

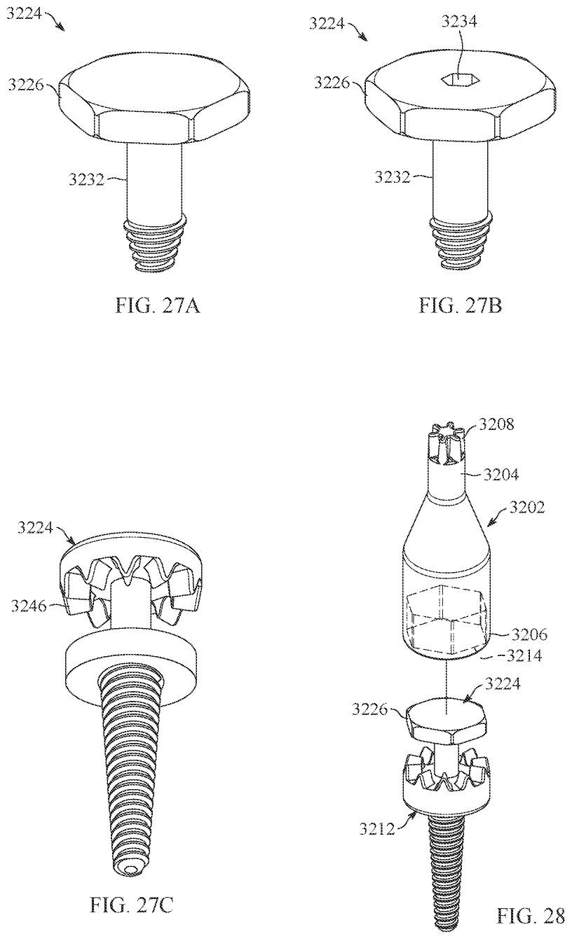

Referring now to FIG. 27a of the present invention illustrating a perspective view of an anti-skidding element 3224 in accordance with an embodiment of the invention. There is shown an anti-skidding element 3224 comprising a longitudinal body 3232 whose one end is centrally attached to the bottom surface of the top 3226. The other end of the longitudinal body 3232 may include a conical shape and has a plurality of threads on its bottom portion, wherein the plurality of threads enables the physical connection between the anti-skidding element 124 and the head 3218 of the fastener 3212. However, the connection between the anti-skidding element 3224 and the head 3218 of the fastener 3212 is not limited to threaded connection, in some embodiments, it may include other connections such as snap and fit connection, magnetic connection, cement or glue retained, and different types of physical connection.

The anti-skidding element 3224 provides intimate and effective engagement between the driving tool 3202 and the fastener 3212 during operation. It allows the sufficient gap for the driving tool 3202, so that plurality of indentations 3208 of the driving tool 3202 and/or the universal jammer 3228 firmly meshes with the plurality of protrusions 3210 of the fastener 3212. The anti-skidding element 3224 maintains the steady rotation of the driving tool 3202 over the head 3218 of the fastener 3212 and eliminates sliding, jumping, or stripping problems associated with fasteners. With the help of the anti-skidding element 3224, an enhanced force of friction and torque is achieved between the fastener 3212 and the driving tool 3202. In the preferred embodiment, the anti-skidding element 3224 includes a hexagonal-shaped top 3226, however, in some embodiments, it may consist of other shapes such as circular, elliptical, square, rectangular, triangular, or polygonal shape as well.

Referring now to FIG. 27C of the present invention illustrating a bottom perspective view of an anti-skidding element 3224 engaged with a fastener in accordance with an alternate embodiment of the present invention. In this embodiment, the anti-skidding element 3224 includes a plurality of indents 3246 provided on the bottom surface of the top. The plurality of indents 3246 is configured to be driven by the plurality of the indentations 3208 of the driving tool 3202, in order to rotate the anti-skidding element 3224. The rotation of the anti-skidding element 3224 causes the engagement of the fastener within the fastening channel or a body. Once the fastening operation is performed, the plurality of indents 3246, when rotated in a reverse direction, further causes the removal of the anti-skidding element 3324 from the head of the fastener.

Referring now to FIG. 28 of the present invention illustrating a removal operation of the anti-skidding element 3224 with the help of the driving tool 3202 in accordance with an embodiment of the present invention. There is shown a driving tool 3202 having a receiving slot 3214 at its bottom portion. The receiving slot 3214 preferably has a shape and size similar to the top 3226 part of the anti-skidding element 3224. In this embodiment, the receiving slot 3214 has a hexagonal shape and is configured to receive the top 3226 part of the anti-skidding element 3224. Thus, both anti-skidding element 3224 and receiving slot 3214 can be of any matching shape to facilitate removing the anti-skidding element 3224 or function as a driver of the fastener 3212 in the opposite direction.

Once the fastening process is performed by the driving tool 3202 and the fastener 3212 is completely inserted within the objects or the fastening channel, then there may or may not be a need for anti-skidding element 3224. It allows the user to adjust the height of the fastener 3212 as per their needs. In such scenarios, the anti-skidding element 3224 is removed by the receiving slot 3214 of the driving tool 3202. The anti-skidding element 3224 can be easily removed from the fastener's head by snapping it using a tool or can be dismantled through threaded connection, friction, magnetic connection, or any other type of connection. In certain embodiments, the anti-skidding element 3224 can also be removed by a hand-driven tool or a motorized tool.

The receiving slot 3214 engages with the top 3226 of the anti-skidding element 3224 and removes it from the head 3218 of the fastener 3212 via a threaded mechanism. In this embodiment, the driving tool 3202 engages with the anti-skidding element 3224 at a 0-degree angle. The driving tool 3202 rotates the anti-skidding element 3224 in a clockwise direction and an anti-clock direction, resulting in the attachment and de-attachment of the anti-skidding element 3224 with the head 3218 of the fastener 3212, respectively. In some embodiments, the anti-skidding element 3224 can also be removed by hex key or Allen key. In these embodiments, the anti-skidding element 3224 (shown in FIG. 27b) includes a hexagonal slot 3234 configured to receive the hex key or Allen key or a specially designed key of any matching shape including triangular, square, rectangle, polygonal, helical, bevel structure, hexagonal and similar; or any tapered configuration thereof.

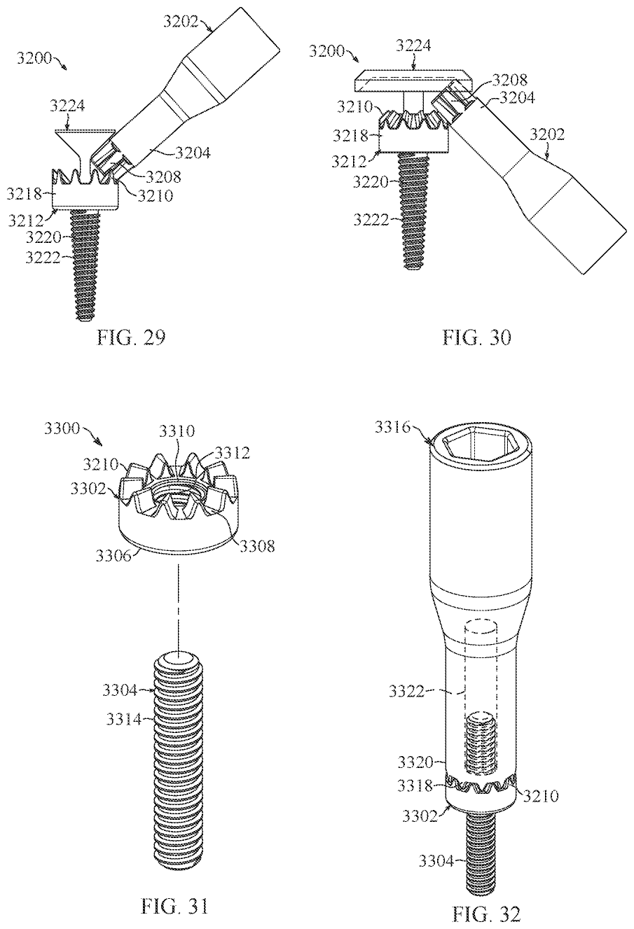

Referring now to FIG. 29 of the present invention illustrating a front view of the fastener 3212 when engaged with the driving tool 3202 at a 45-degree angle. In this embodiment, the anti-skidding element 3224 of the fastener 3212 has a different configuration and structure than the element discussed for 90-degree engagement. It is also important to note that the fastener 3212, the head 3218, and the anti-skidding element 3224 are detachable from one another in some embodiments. In some embodiments and applications, two or all three components are one unit. The top 3226 of the anti-skidding element 3224 is positioned away from the longitudinal body 3232, and is structured in a way to allow the smooth engagement of the driving tool 3202 with the fastener 3212. Referring now to FIG. 30 of the present invention illustrating a front view of the fastener 3212 when engaged with the driving tool 3202 at a 135-degree angle. In this embodiment, the top 3226 of the anti-skidding element 3224 is positioned towards the longitudinal body 3232 and is structured to facilitate the smooth engagement of the driving tool 3202 with the fastener 3212. As described above, the same depicted driving tool 3202 with head configuration can be utilized for the angled driving embodiments and approaches.

Referring now to FIG. 31 of the present invention illustrating a universal nut and bolt assembly 3300 in accordance with an embodiment of the present invention. There is shown a universal nut 3302 and a bolt 3304 when in the disengaged position. The universal nut 3302 and bolt 3304 are made of any solid material such as metals, ceramics, plastics, rubbers, silicones, cast iron, aluminum alloy, titanium, ABS (Acrylonitrile Butadiene Styrene), polycarbonate, etc., offering good strengthening properties during mechanical operations. The universal nut 3302 is configured to be attached with the bolt 3304 to provide a clamping force and prevent axial movement.

The universal nut 3302 comprises a circular body with a top surface 3308 and a bottom surface 3306, wherein the top surface 3308 includes a plurality of protrusion 3210 positioned circumferentially over the surface. In a preferred embodiment, the plurality of protrusion 3210 has a V-shaped structure. However, the plurality of protrusion 3210 is not limited to a V-shaped configuration. It may include other shapes, such as U-shape, Wave-shape, helical, cycloidal, bevel, S-shaped, wave-shaped, circular, square, rectangular, triangular, hexagonal, polygonal shape, and similar; or any tapered configurations thereof. During operation, the plurality of protrusion 3210 meshes with the plurality of indentations 3208 of the driving tool 3202 causes the rotation of the universal nut 3302 over the bolt 3304. Thus, the universal nut 3302 can interact and mesh with fasteners and can be designed to match any fastener and driver to fulfill its objectives. The universal nut 3302 further includes a threaded hole 3310 comprising a plurality of internal threads 3312 on its inner portion. The plurality of internal threads 3312 engages with a plurality of external threads 3314 when the universal nut 3302 is rotated by the driving tool 3202. The clockwise rotation of the universal nut 3302 over the bolt 3304 results in the tightening or clamping of the fastener, and the anticlockwise rotation of the universal nut 3302 results in the loosening of the fastener. The universal nut 3302 preferably has a round shape, however, in some embodiments, it may include other shapes such as triangular, hexagonal, square, rectangular, oval, elliptical, and polygonal shape as well.

Referring now to FIG. 32 of the present invention illustrating an engagement between the driving tool 3316 and the universal nut 3302 in accordance with an alternative embodiment. There is shown a driving tool 3316 includes a plurality of indentations 3318 on its one end and a tool head 3320 on its other end. The driving tool 3316 further includes a recess 3322 for receiving the bolt 3304. The recess 3322 can be through-and-through or open for the entire length of the tool or can have a stop in the shaft depending on the application. In some embodiments, the recess 3322 may receive the different types of threaded fasteners such as screws. During the fastening operation, the plurality of indentations 3318 of the driving tool 3316 firmly engages with the plurality of protrusion 3210 of the universal nut 3302. When the driving tool 3316 rotates the universal nut 3302 in the clockwise direction, the universal nut 3302 rotates over the length of the bolt 3304. Further, the rotation of the universal nut 3302 over the bolt 3304 causes the insertion of bolt 3304 within the recess 3322 of the driving tool 3316, and desired fastening between two or more objects is achieved. The recess 3322 also functions as a guide for the universal nut 3302. Furthermore, the driving tool 3316 also helps in removing the universal nut 3302 when rotated in the anti-clockwise direction.

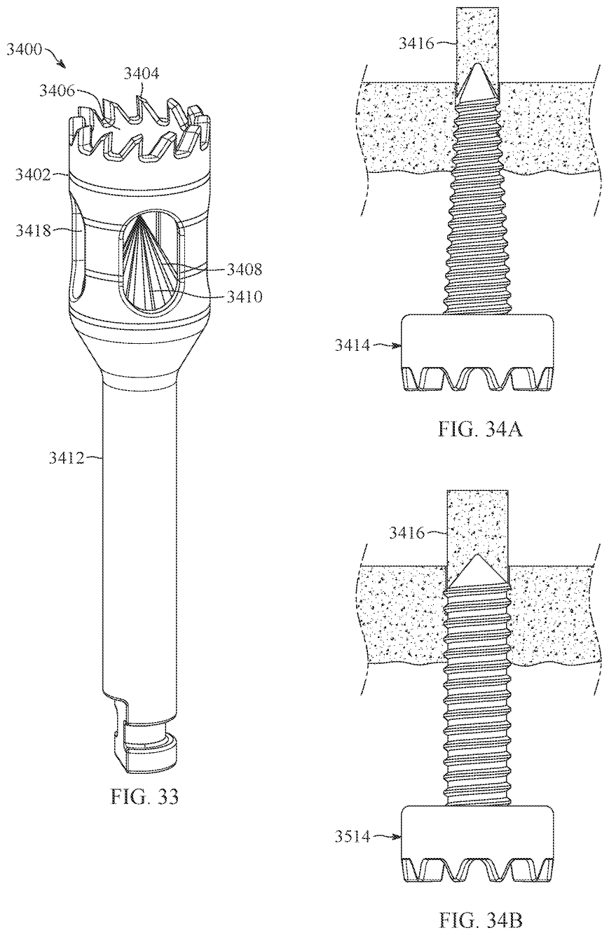

Referring now to FIG. 33 of the present invention illustrating a universal lifter 3400 in accordance with an embodiment of the invention. The universal lifter 3400 performs the drilling operation and helps in pushing or lifting the material to a certain extent. In scenarios where a material needs to be lifted in order to perform a specific task, for example, in dentistry, the sinus membrane is lifted to increase the amount of dental bone, the universal lifter 3400 can be employed. The universal lifter 3400 can also be employed in various industries such as medical, construction industry, transportation, electrical, architecture, aerodynamics, robotics, automotive, infrastructure, agriculture, military, etc. Based on the requirement, the universal lifter 3400 lifts the material horizontally and vertically. As shown in FIG. 33, the universal lifter 3400 includes a hollow cylindrical body 3402 comprising a plurality of cutting blades 3404 at its one end. The cylindrical body 3402 includes a bore 3406 extending through to separate the material, which is circumferentially cut by the plurality of cutting blades 3404. In this embodiment, the plurality of cutting blades 3404 has an inclined structure and is configured to cut the material circumferentially when rotated in a clockwise direction. Although, the plurality of cutting blades 3404 does not cut the material when turned in an anti-clockwise rotation. In some embodiments, the plurality of cutting blades 3404 may include standard tooth blades, skip-tooth blades, double-tooth blades, reverse skip-tooth blades, precise-guide blades, and wave-like, v-like, u-like, triangular, square, rectangular, rounded, trapezoid, and polygonic shape blades.

The plurality of cutting blades 3404 can be made of any solid materials, including metals such stainless steel, cobalt, and carbide material, providing high strength during material cutting. In some embodiments, especially in the medical field, the plurality of cutting blades 3404 is made of non-reactive, inert, or biocompatible surgical material such as titanium, stainless steel, etc. The plurality of cutting blades 3404 is sharp enough to cut a wide variety of materials, including rigid and flexible materials such as plastic, iron, wood, concrete, aluminum, copper, and the like. The material can be a bone, gums, membranes, and muscle if the universal lifter 3400 is applied in the medical and surgical industry.

The universal lifter 3400 further includes a drill bit 3408 configured to perform drilling operation over the material that is circumferentially cut by the plurality of cutting blades 3404. The drill bit 3408 is centrally positioned on the distal end of the cylindrical body 3402. The drill bit 3408 includes a plurality of blades 3410 that cut or grind the material thoroughly and make a sufficient space to receive a fastener. The drill bit 3408 can be made of any solid material such as metals, for example, low and high carbon steel, to perform better drilling operations. In some embodiments, the drill bit 3408 can also be made of cobalt, carbide, tungsten carbide, and polycrystalline diamond, offering high strength during the drilling operation. The cylindrical body 3402 further includes a plurality of openings 3418 through which material 3416 will come out when the drilling operation is performed or when the universal lifter 3400 is reversed out of the complex. The cylindrical body 3402 and the drill bit 3408 are configured to be rotated by a driving shaft 3412 positioned at the other end of the universal lifter 3400. In a preferred embodiment, the driving shaft 3412 is rotated by a motorized device such as a motorized latch driver. In some embodiment, the driving shaft 3412 can also be rotated by the hand-driven tool or driven with other motorized tools that can be made to match the configuration or shape of the driving shaft 3412.

The universal lifter 3400 may have a plurality of markings 3420 anywhere on the cylindrical body 3402 of the universal lifter 3400. The plurality of markings 3420 may include demarcations, indentations, or colorations used for measurement purposes. The plurality of markings 3420 indicates the value (length, distance) to which the universal lifter 3400 is inserted within the material. The plurality of markings 3420 is created to inform the operator how much of the universal lifter 3400 has advanced into the material and at what stage it can be reversed after it reaches its destination.

During operation, when the universal lifter 3400 is rotated by the handheld or motorized tool, the plurality of cutting blades 3404 cut the material circumferentially. Thereafter a short distance is created for the core, the drill bit 3408 starts the drilling operation over the material and drills in such a way that it pushes the cut-out material towards the walls via the plurality of openings 3418, and side-by-side, it densifies the adjacent material presents near the wall. The drill bit 3408 makes the hole in the material and prepares the site to receive a fastener 3414, such as a tapered screw or bolt-like fastener. The screw-like fastener 3414 is illustrated in FIG. 34a, and bolt-like fastener 3514 is shown in FIG. 34b, which lifts the material 3416. In some embodiments, the head of the fasteners (3414, 3514) may contact the material's lower surface, and their protrusions are still available for driving purposes. At this stage, the fasteners (3414, 3514) can also act as a connection point for other bodies and side by side controlling the pushing or lifting of the material.

Once the drilling operation is completed and the universal lifter 3400 is reversed out and removed, the circumferential cut-out material is left within the channel Thereafter, a fastener 3414 is secured to the hole made by the drill bit 3408 through a threaded mechanism. FIG. 34a and FIG. 34b illustrate the engagement between the fasteners (3414, 3514) and the material 3416 cut or drilled by the universal lifter 3400. The cut-out portion guides the fastener 3414 as it rotates. The fastener 3414 engaged with the densified sides and produced the forces needed to lift the material 3416. The fastener 3414 can be engaged with the material via a driving tool mentioned in the above description of the application. When the fastener 3414 is rotated by the driving tool, the material 3416 starts lifting or pushing to the desired height.

As the fastener 3414 is engaged with the large area of the material 3416, it lifts the material 3416 through its rotational movement or force. In this embodiment, the material 3416 does not completely leave the complex. In other embodiments where an elastic material supports material 3416, the fastener 3414 can continue to be driven up in a controlled fashion as not to damage the elastic support. For example, in an alternative oral sinus lift protocol, the material 3416 can further dissociate from the bone floor complex while the core membrane stays intact and continue the lift without rupturing the supporting membrane. In that embodiment, the fastener 3414 can lift the material 3416 until it reaches stability. In some embodiments, the drill bit 3408 may be a rounded tip to perform the pushing while simultaneously cutting operation is performed by the plurality of cutting blades 3404. In other embodiments, the drill bit 3408 may be removed to collect or harvest a core versus leaving it behind for different applications.

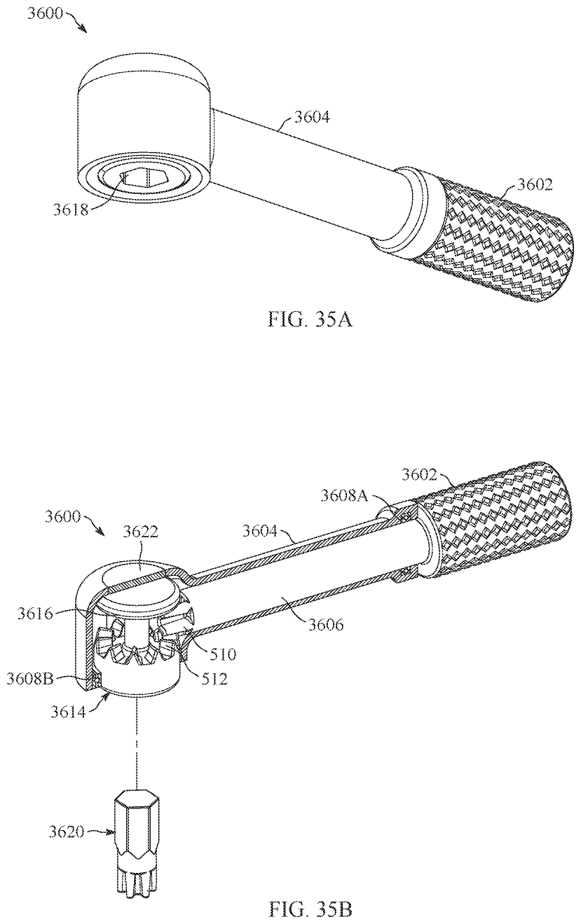

Referring now to FIG. 35a of the present invention illustrating a perspective view of handheld universal driver 3600 in accordance with an embodiment of the invention. There is shown a handheld universal driver 3600 configured to fasten and unfasten a plurality of fasteners such as a bolt, screw, nuts, start head, and hex head fasteners, and is able to rotate about 360-degree. The user can also utilize the handheld universal driver 3600 as a wrench tool. It can have the same motion as the wrench tool and rotates 360-degree to fasten and unfasten the plurality of fasteners. The handheld universal driver 3600 can be used in all fastening applications as discussed in the above embodiments of the inventions. In addition, the situations where the fastening of objects is impossible due to less space and the areas where the heads of the fastener are not accessible for driving purposes, the handheld universal driver 3600 can be employed. The handheld universal driver 3600 is made of any solid material such as cast iron, titanium, metal, solid material, aluminum, alloy, brass, bronze, and any other material having high strengthening features. The handheld universal driver 3600 can be a single piece or may comprise a plurality of components and parts that are detachably attached, allowing the repairing and replacement of the components when needed. The handheld universal driver 3600 is configured to be driven by a battery or a power source. In one embodiment, the handheld universal driver 3600 can also be driven by a user manually.

The handheld universal driver 3600 comprises a handle 3602, which may have a patterned surface that allows the user to hold or grip the driver more efficiently. The handle 3602 is rotatably attached to a housing 3604 shown in FIG. 35a and structured in a way to facilitate the rotation of the handle 3602 while the housing 3604 remains fixed in its position. In some embodiments, a bearing component 3608A is placed between the handle 3602 portions and the housing 3604. More specifically, the bearing component 3608A allows the 360-degree rotation of the handle 3602, and the housing 3604 remains fixed due to the force of friction. However, the connection between the handle 3602 and the housing 3604 is not limited to the bearing mechanism, it may include other mechanisms such as a ratchet mechanism or a force amplifying mechanism that increase the output force and eases the rotation of the handle 3602.

Referring now to FIG. 35b of the present invention illustrating the internal components of the handheld universal driver 3600. The handle 3602 includes a driving rod 3606, whose one end is fixed to the handle 3602 and rotates in the same direction in which the handle 3602 is rotated. In addition, the handle 3602 includes a plurality of indentations 3610 at one end that engages with the plurality of protrusions 3612 of an adaptor 3614 positioned in the housing 3604 of the handheld universal driver 3600. During engagement, with the help of the handle 3602, the driving rod 3606 rotates in the horizontal plane, further causing the rotation of the adaptor 3614 in the vertical plane. The adaptor 3614 has a similar configuration as the fastener 3212 disclosed in the above embodiments of the invention. The adaptor 3614 may also include an anti-skidding element 3616 that maintains the steady rotation of driving rod 3606 over the plurality of protrusions 3612.

In a preferred embodiment, the adaptor 3614 further includes a hexagonal opening 3618 configured to receive a bit 3620 rotated by the handheld universal driver 3600. However, in alternate embodiments, the adaptor may include a circular, square, rectangular, polygonal, triangular opening to receive a matching bit 3620. The bit 3620 firmly engages with the adaptor 3614 through a snap and fit mechanism. It may include a magnetic mechanism, a friction mechanism, or another mechanism for engagement purposes. The bit 3620 turns in the same direction in which the adaptor 3614 is rotated. A bearing component 3608B may also be provided in handheld universal driver 3600 that eases the rotation of the adaptor 3614 and eliminates the friction between the moving parts. The clockwise and anti-clockwise rotation of the bit 3620 by the handheld universal driver 3600 causes the tightening and loosening of the fastener, respectively. Further, the bottom end of the bit 3620 engages with the head of the fastener for driving purposes. In one embodiment, the bit 3620 has a hexagonal structure that matches with the configuration of the hexagonal opening 3618, however in alternate embodiments, it is designed to engage with a bit 3620 of different sizes and shapes such as circular, square, rectangular, triangular, hexagonal, polygonal shape, and similar; or any tapered configurations thereof.

In one embodiment, the handheld universal driver 3600 may employ a detachable cover that allows the replacement of the adaptor 3614 when needed. In this embodiment, the handheld universal driver 3600 includes a cover 3622 detachably attached to the housing 3604 through a connecting mechanism. The cover 3622 can be attached to the housing 3604 through a snap and fit mechanism, magnetic mechanism, threaded mechanism, etc. The cover 3622 is provided above the adaptor 3614 and can be removed when replacement is needed. For replacement, the handle 3602, which is permanently attached to the driving rod 3606, moves backward, causing the disengagement of the driving rod 3606 and the adaptor 3614. Then, the cover 3622 is detached from the housing 3604, and the adaptor 3614 is removed or replaced.

According to an alternative embodiment, the handheld universal driver 3600 may also include a universal jammer 3228 (disclosed in the above embodiments of the invention) comprising a plurality of indentations 3208 that engages with the plurality of protrusions 3612 of the adaptor 3614. In this embodiment, the driving rod 3606 may include a hexagonal structure at one end instead of the plurality of indentations. During operation, the hexagonal end of driving rod 3606 engages with the respective receiving of the universal jammer 3228. The rotational motion of the universal jammer 3228 by the driving rod 3606 in the horizontal plane further causes the rotation of the adaptor 3614 in the vertical plane.

What is claimed is:

1. A fastening system comprising:
a fastener comprising a head having a plurality of protrusions, and a shank centrally attached with the head and having a plurality of male threads allowing an insertion of the fastener within a fastening channel; and
a driving tool comprising a first end and a second end connected through a cylindrical shaft, the first end is a tip end having a plurality of indentations that facilitates an engagement of driving tool with the plurality of protrusion on the fastener at different angles.

2. The fastening system of claim 1, wherein the second end of the driving tool is a tool head for receiving a driving shaft rotatable by a hand-driven tool, a motorized tool, or a latch driver.

3. The fastening system of claim 1, wherein shape of the plurality of indentations and the plurality of protrusions includes but is not limited to V-shape, cycloidal shape, worm-gear shape, herringbone gear shape, and spur gear shape.

4. The fastening system of claim 1, wherein the shank and the head of the fastener can be detachably attached with each other by means of a snap and fit mechanism, threaded mechanism, magnetic mechanism, or any other connecting mechanism.

5. The fastening system of claim 1, wherein the fastener is selected from a group containing non-permanent fasteners, wherein non-permanent fasteners include but are not limited to screw fasteners, bolt fasteners, or any other threaded fasteners.

6. The fastening system of claim 1, wherein the driving tool when engaged, a clockwise and anticlockwise rotation of the driving tool causes the fastener to rotate accordingly.

7. The fastening system of claim 1, wherein the head of the fastener has a tapered shape comprising a plurality of protrusions or teeth which are positioned or arranged in a way to facilitate engagement of the driving tool with the fastener at different angles.

8. The fastening system of claim 1, wherein the fastener further comprises a support element that supports an object placed over the fastener, the object is being employed to cover a whole portion of the fastener.

9. The fastening system of claim 8, wherein the support element comprises a receiving slot for receiving an extruded part of the object, causing a proper fixation of the object over the support element.

10. The fastening system of claim 1, wherein the fastener comprises a movable adaptor provided on the shank of the fastener.

11. The fastening system of claim 10, wherein the movable adaptor has a hexagonal structure at base and semi-circular structure from top.

12. The fastening system of claim 11, wherein the movable adaptor is mechanically driven about central axis of the fastener.

13. The fastening system of claim 1, wherein the fastener is mounted inside an object.

14. The fastening system of claim 13, wherein the object comprises a recessed portion that receives the head of the fastener.

15. The fastening system of claim 13, wherein a covering member is non-permanently fixed to the object by means of a snap, friction, locking, cement, magnetic, glue, resin, or any other connecting mechanism.

16. The fastening system of claim 1 further comprises an anti-skidding element on top of the head of the fastener.

17. The fastening system of claim 16, wherein the anti-skidding element is detachably attached with the head of the fastener.

18. The fastening system of claim 1, wherein the driving tool further comprises a protrusion member that extends centrally from the tip end.

19. The fastening system of claim 18, wherein the protrusion member gets engaged with a receiving slot located centrally on the head of the fastener.

20. The fastening system of claim 18, wherein the protrusion member is in hexagonal, V-shape structure, helical structure, cycloidal structure, bevel structure, S-shaped structure, wave-shaped, circular, square, rectangular, triangular, polygonal shape and similar; or any tapered configurations thereof.

21. The fastening system of claim 19, wherein the protrusion member can be located on the head of the fastener and the tip end of the driving tool.

22. The fastening system of claim 1 further comprises a jammer having a plurality of indentations that engages with the plurality of protrusions of the fastener at multiple angles.

23. The fastening system of claim 1, wherein the tip end of the driving tool is used as a jammer.

24. The fastening system of claim 22, wherein a bottom portion of the jammer comprises a groove configured to receive a drive shaft.

25. The fastening system of claim 1 further comprises a nut having a circular body with a top surface and a bottom surface.

26. The fastening system of claim 25, wherein the nut comprises a threaded hole that facilitates rotation of the nut over the fastener.

27. The fastening system of claim 1 further comprises a lifter configured to cut a material circumferentially and drills the material centrally.

28. The fastening system of claim 27, wherein the lifter is used to vertically or horizontally lift the material to a desired height or length.

29. The fastening system of claim 27, wherein the lifter comprises a cylindrical body having a plurality of cutting blades positioned on one end of the cylindrical body.

30. The fastening system of claim 29, wherein the cylindrical body comprises a bore extending axially for receiving the material cut by the cutting blades.

31. The fastening system of claim 27 further comprises a drill bit.
